(19) United States
(12) Patent Application Publication
(10) Pub. No.: US 2023/0201326 A1
(43) Pub. Date: Jun. 29, 2023

(54) Immunogenic Compositions and Vaccines in the Treatment and Prevention of Infections

(71) Applicant: Longhorn Vaccines and Diagnostics, LLC, Bethesda, MD (US)

(72) Inventors: Gerald W. Fischer, Bethesda, MD (US); Clara J. Sei, Germantown, MD (US)

(73) Assignee: Longhorn Vaccines and Diagnostics, LLC, Bethesda, MD (US)

(21) Appl. No.: 17/985,296

(22) Filed: Nov. 11, 2022

Related U.S. Application Data
(60) Provisional application No. 63/333,780, filed on Apr. 22, 2022.
(60) Provisional application No. 63/278,759, filed on Nov. 12, 2021.

(51) Int. Cl. A61K 39/116; A61K 39/04; A61K 47/64

(52) U.S. Cl. CPC A61K 39/116; A61K 39/04; A61K 47/646

(57) ABSTRACT

The invention is directed to portions of proteins of gram-positive bacteria, gram-negative, acid-fast bacteria (Mycobacteria, Staphylococcus) and/or virus (SARS-COV-2, Influenza), and antibodies reactive against these portions that can be formulated as immunogenic compositions and vaccines for the treatment and prevention of a microbial and/or viral infections. Preferably, compositions of the invention contain one or more portions of selected microbial and/or viral proteins that, upon administration to a subject, generate an effective cellular and/or humoral immune response, modulate immunity and a cytokine response. Effective responses involve an increased generation of antibodies that enhance immunity against an infection and promote an enhanced a phagocytic response. Monoclonal antibodies produced against these peptides enhance phagocytosis and killing of bacteria, viruses, and other microbes by phagocytic cells, and enhance clearance from the blood.

### REFERENCE TO RELATED APPLICATIONS

This application claims priority to U.S. Provisional Application No. 63/333,780 filed Apr. 22, 2022, and U.S. Provisional Application No. 63/278,759 filed Nov. 12, 2021, the entirety of each of which is incorporated by reference.

### SEQUENCE LISTING

The instant application contains a Sequence Listing which has been submitted electronically in ASCII format and is hereby incorporated by reference in its entirety.

### BACKGROUND

### 1. Field of the Invention

The present invention is directed to peptides, compositions, vaccines, and methods for treating and preventing diseases and/or disorder associated with Mycobacterial infection, and also for enhancing the immune system of a patient against other microbial infections such as gram positive and negative bacteria and viruses, and other disorders. In particular, peptides, compositions, vaccines, and methods that relate to treating and preventing infection by multidrug resistant (MDR), extremely drug resistant (XDR), and latent Mycobacterial infection such as infection of Mycobacterium tuberculosis.

### 2. Description of the Background

Mycobacterium tuberculosis (MTB) is a pathogenic bacterial species in the family Mycobacteriaceae and the causative agent of most cases of tuberculosis (TB). Another species of this genus is M. leprae, the causative agent of leprosy. MTB was first discovered in 1882 by Robert Koch, M. tuberculosis has an unusual, complex, lipid rich, cell wall which makes the cells impervious to Gram staining. Acid-fast detection techniques are used to make the diagnosis instead. The physiology of M. tuberculosis is highly aerobic and requires significant levels of oxygen to remain viable. Primarily a pathogen of the mammalian respiratory system, MTB is generally inhaled and, in five to ten percent of individuals, will progress to an acute pulmonary infection. The remaining individuals will either clear the infection completely or the infection may become latent. It is not clear how the immune system controls MTB, but cell mediated immunity is believed to play a critical role (Svenson et al., Human Vaccines, 6-4:309-17, 2010). Common diagnostic methods for TB are the tuberculin skin test, acid-fast stain, and chest radiographs.

Well over ninety percent of individuals infected with MTB remain outwardly healthy with no demonstrable symptoms. These individuals are classified as latently infected and are a reservoir from which active MTB cases continue to develop (“reactivation tuberculosis”). Latent infection is generally defined as the absence of clinical symptoms of TB in addition to a delayed hypersensitivity reaction to the purified protein derivative of MTB used in tuberculin skin test or a T-cell response to MTB-specific antigens. The absence of an understanding of latency and thereby reliable control measures for treatment, makes latent tuberculosis infections a serious problem.

M. tuberculosis requires oxygen to proliferate and does not retain typical bacteriological stains due to high lipid content of its cell wall. While mycobacteria do not fit the Gram-positive category from an empirical standpoint (i.e., they do not retain the crystal violet stain), they are classified as acid-fast Gram-positive bacteria due to their lack of an outer cell membrane.

M. tuberculosis has over one hundred strain variations and divides every 15-20 hours, which is extremely slow compared to other types of bacteria that have division times measured in minutes (e.g., Escherichia coli can divide roughly every 20 minutes). The microorganism is a small bacillus that can withstand weak disinfectants and survive in a dry state for weeks. The cell wall of MTB contains multiple components such as peptidoglycan, mycolic acid, and the glycolipid lipoarabinomannan. The role of these moieties in pathogenesis and immunity remain controversial. (Svenson et al., Human Vaccines, 6-4:309-17, 2010).

MTB infection is spread most typically by airborne droplets, which contain the pathogen expelled from the lungs and airways of those with active or otherwise infectious TB. The infectious droplets are inhaled and lodge in the alveoli and in the alveolar sac where M. tuberculosis is taken up by alveolar macrophages. These macrophages invade the subtending epithelial layer, which leads to a local inflammatory response initiating formation of the granuloma, the hallmark of tuberculosis disease. That results in recruitment of mononuclear cells from neighboring blood vessels, thus providing fresh host cells for the expanding bacterial population. However, these macrophages are unable to digest the bacteria because the cell wall of the bacteria prevents the fusion of the phagosome with a lysosome. Specifically, M. tuberculosis blocks the bridging molecule, early endosomal autoantigen 1 (EEA1); however, this blockade does not prevent fusion of vesicles filled with nutrients. As a consequence, bacteria multiply unchecked within the macrophage. The bacteria also carry the UreC gene, which prevents acidification of the phagosome which allows the bacterium to evade macrophage-killing by neutralizing reactive nitrogen intermediates.

With the arrival of lymphocytes, the granuloma acquires a more organized, stratified structure. Development of an immune response takes about 4 to 6 weeks after the primary infection is indicated by a positive DTH (delayed type hypersensitivity) reaction to Tuberculin. The balance between host immunity (protective and pathologic) and bacillary multiplication determines the outcome of infection. An encounter with MTB is classically regarded to give rise to three possible outcomes. The first possible outcome, which occurs in a minority of the population, is the rapid development of active TB and associated clinical symptoms. The second possible outcome, which occurs in the majority of infected individuals, do not include disease symptoms. These individuals develop an effective acquired immune response and are considered to have a “latent infection.” A portion of latently infected individuals over time will reactivate and develop active TB. Roughly ten percent of these infected individuals (mainly infants or children) will develop progressive clinical disease referred to as primary or active TB. Primary TB usually occurs within 1-2 years after the initial infection. This results from local bacillary multiplication and spread in the lung and/or blood. Spread through the blood can seed bacilli in various tissues and organs. Post-primary TB, or secondary TB, can occur many years after infection owing to loss of immune control and the reactivation of bacilli. The immune response of the patient results in a pathological lesion that is characterized by localized, often extensive tissue damage, and cavitations. The characteristic features of active post-primary TB can include extensive lung destruction with cavitation, positive sputum smear (most often), and upper lobe involvement; however these are not exclusive. Patients with cavitary lesions (i.e., granulomas that break through to an airway) are the main transmitters of infection. In latent TB, the host immune response is capable of controlling the infection but falls short of eradicating the pathogen. Latent TB is defined solely on the evidence of sensitization by mycobacterial proteins that is a positive result in either the Tuberculin skin test (TST) reaction to purified protein derivative of MTB or an in vitro interferon-gamma (IFN-γ) release assay to MTB-specific antigens, in the absence of clinical symptoms or isolated bacteria from the patient.

The BCG vaccine (Bacille de Calmette et Guérin) against tuberculosis is prepared from a strain of the attenuated, but live bovine tuberculosis bacillus, Mycobacterium bovis. This strain lost its virulence to humans through in vitro subculturing in Middlebrook 7H9 media. As the bacteria adjust to subculturing conditions, including the chosen media, the organism adapts and in doing so, loses its natural growth characteristics for human blood. Consequently, the bacteria can no longer induce disease when introduced into a human host. However, the attenuated and virulent bacteria retain sufficient similarity to provide immunity against infection of human tuberculosis. The effectiveness of the BCG vaccine has been highly varied, with an efficacy of from zero to eighty percent in preventing tuberculosis for duration of fifteen years, although protection seems to vary greatly according to geography and the lab in which the vaccine strain was grown. This variation, which appears to depend on geography, generates a great deal of controversy over use of the BCG vaccine yet has been observed in many different clinical trials. For example, trials conducted in the United Kingdom have consistently shown a protective effect of sixty to eighty percent, but those conducted in other areas have shown no or almost no protective effect. For whatever reason, these trials all show that efficacy decreases in those clinical trials conducted close to the equator. In addition, although widely used because of its protective effects against disseminated TB and TB meningitis in children, the BCG vaccine is largely ineffective against adult pulmonary TB, the single most contagious form of TB.

A 1994 systematic review found that the BCG reduces the risk of getting TB by about fifty percent. There are differences in effectiveness, depending on region due to factors such as genetic differences in the populations, changes in environment, exposure to other bacterial infections, and conditions in the lab where the vaccine is grown, including genetic differences between the strains being cultured and the choice of growth medium.

The duration of protection of BCG is not clearly known or understood. In studies showing a protective effect, the data are inconsistent. The MRC study showed protection waned to 59% after 15 years and to zero after 20 years; however, a study looking at Native Americans immunized in the 1930s found evidence of protection even 60 years after immunization, with only a slight waning in efficacy. Rigorous analysis of the results demonstrates that BCG has poor protection against adult pulmonary disease but does provide good protection against disseminated disease and TB meningitis in children. Therefore, there is a need for new vaccines and vaccine antigens that can provide solid and long-term immunity to MTB.

The role of antibodies in the development of immunity to MTB is controversial. Current data suggests that T cells, specifically CD4⁺ and CD8⁺ T cells, are critical for maximizing macrophage activity against MTB and promoting optimal control of infection (Slight et al, JCI 123(2):712, February 2013). However, these same authors demonstrated that B cell deficient mice are not more susceptible to MTB infection than B cell intact mice suggesting that humoral immunity is not critical. Phagocytosis of MTB can occur via surface opsonins, such as C3, or nonopsonized MTB surface mannose moieties. Fc gamma receptors, important for IgG facilitated phagocytosis, do not seem to play an important role in MTB immunity (Crevel et al., Clin Micro Rev. 15(2), April, 2002; Armstrong et al., J Exp Med. 1975 Jul. 1; 142(1):1-16). IgA has been considered for prevention and treatment of TB, since it is a mucosal antibody. A human IgA monoclonal antibody to the MTB heat shock protein HSPX (HSPX) given intranasally provided protection in a mouse model (Balu et al., J of Immun. 186:3113, 2011). Mice treated with IgA had less prominent MTB pneumonic infiltrates than untreated mice. While antibody prevention and therapy may be hopeful, the effective MTB antigen targets and the effective antibody class and subclasses have not been established (Acosta et al, Intech, 2013).

Cell wall components of MTB have been delineated and analyzed for many years. Lipoarabinomannan (LAM) has been shown to be a virulence factor and a monoclonal antibody to LAM has enhanced protection to MTB in mice (Teitelbaum, et al., Proc. Natl. Acad. Sci. 95:15688-15693, 1998, Svenson et al., Human Vaccines, 6-4:309-17, 2010). The mechanism whereby the MAB enhanced protection was not determined, and the MAB did not decrease bacillary burden. It was postulated that the MAB possibly blocked the effects of LAM induced cytokines. The role of mycolic acid for vaccines and immune therapy is unknown. It has been used for diagnostic purposes but has not been shown to have utility for vaccine or other immune therapy approaches. While MTB infected individuals may develop antibodies to mycolic acid, there is no evidence that antibodies in general, or specifically mycolic acid antibodies, play a role in immunity to MTB.

Antibiotic resistance is becoming more and more of a problem for treating MTB infections. Beginning with the first antibiotic treatment for TB in 1943, some strains of the TB bacteria developed resistance to the standard drugs through genetic changes. The BCG vaccine against TB does not provide protection from acquiring TB to a significant degree. In fact, resistance accelerates if incorrect or inadequate treatments are used, leading to the development and spread of multidrug-resistant TB (MDR-TB). Incorrect or inadequate treatment may be due to use of the wrong medications, use of only one medication (standard treatment is at least two drugs), not taking medication consistently or for the full treatment period (treatment is generally required for several months). Treatment of MDR-TB requires second-line drugs (e.g., fluoroquinolones, aminoglycosides, and others), which in general are less effective, more toxic, and much more expensive than first-line drugs. If these second-line drugs are prescribed or taken incorrectly, further resistance can develop leading to extreme-drug resistant TB (XDR-TB). Resistant strains of TB are already present in the population, so MDR-TB and XDR-TB are directly transmitted from an infected person to an uninfected person. Thus, a previously untreated person can develop a new case of MDR-TB or XDR-TB absent in prior infection and/or treatments. This is known as primary MDR-TB or XR-TB and is responsible for up to 75% of new TB cases. Acquired MDR-TB and XR-TB develops when a person with a non-resistant strain of TB is treated inadequately, resulting in the development of antibiotic resistance in the TB bacteria infecting them. These people can in turn infect other people with MDR-TB.

Drug-resistant TB caused an estimated 480,000 new TB cases and 250,000 deaths in 2015, and accounts for about 3.3% of all new TB cases worldwide. These resistant forms of TB bacteria, either MDR-TB or rifampin-resistant TB, cause 3.9% of new TB cases and 21% of previously treated TB cases. Globally, most drug-resistant TB cases occur in South America, Southern Africa, India, China, and areas of the former Soviet Union.

Treatment of MDR-TB requires treatment with second-line drugs, usually four or more anti-TB drugs for a minimum of 6 months, and possibly extending for 18 to 24 months if rifampin resistance has been identified in the specific strain of TB with which the patient has been infected. Under ideal program conditions, MDR-TB cure rates can approach 70%. XR-TB infection requires even more-robust and prolonged treatment regimens.

Thus, there is a strong need to provide or improve products and approaches to prevent and treat microbial diseases including but not limited to bacterial and viral infections.

### SUMMARY OF THE INVENTION

The present invention overcomes the problems and disadvantages associated with current strategies and designs and provide new tools and methods for treating or preventing a microbial infection and enhancing the immune system of a patient.

One embodiment of the invention is directed to peptides which include peptide mimotopes (or simple mimotopes), and portions of peptides and mimotopes obtained or derived from a microbe such as a Mycobacteria species or another gram-positive (e.g., S. aureus), a gram-negative bacteria (e.g. E. coli), or a virus (e.g., influenza, corona virus). Preferably the peptide comprises a portion or a mimotope of peptidoglycan, a heat shock protein, mycolic acid, lipoteichoic acid, lipoarabinomannan, or a Mycobacterial or other gram-positive bacterial surface antigen. Peptides of the invention include composite peptides and mimotopes, fusion peptides, peptide conjugates, and synthetic sequence. Also preferably, the peptide comprises one or more of the sequences of SEQ ID NOs. 1-41.

Preferably, an immunogenic peptide of this disclosure is comprised of a contiguous sequence of any one of the sequences of SEQ ID NOs. 1-4, 18-24, or a combination thereof. Preferably the contiguous sequence further includes one or more of the sequences selected from the group consisting of the sequences of SEQ ID NOs. 5-17 and 25-41. Also preferably, an immunogenic peptide of this disclosure is comprised of a contiguous sequence of any one of the sequences of SEQ ID NOs. 25, 30, 32, 36, 38, 39, 41, or a combination thereof, or a combination thereof. Preferably the contiguous sequence further includes one or more of the sequences selected from the group consisting of the sequences of SEQ ID NOs. 1-24, 26-29, 31, 33-35, 37, and 40.

Preferably the peptides disclosed herein contain a sequence of a viral antigen, a bacterial antigen, a parasitic antigen, a composite antigen, or a combination thereof. Preferably, the bacterial antigen comprises an antigen of a gram-positive microorganism, a gram-negative microorganism, both gram-positive and gram-negative microorganisms, or an acid-fast microorganism and, preferably contains the sequence of a T-cell stimulating epitope and/or a composite epitope, which may be a bacterial or viral epitope.

Another embodiment of the invention comprises immunogenic compositions comprising the peptides disclosed herein. Preferably, the immunogenic compositions are comprised of one or more of a pharmaceutically acceptable carriers, a chemical agent, a diluent, an excipient, or an adjuvant. Preferred pharmaceutically acceptable carriers include chemical agent, diluent, or excipient comprises water, fatty acids, lipids, polymers, carbohydrates, gelatin, solvents, saccharides, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, or a combination thereof. Preferred adjuvants comprise alum, oil in water emulsion, amino acids, proteins, carbohydrates, Freund’s, a liposome, saponin, lipid A, squalene, liposomes adsorbed to aluminum hydroxide, liposomes containing QS21 saponin, liposomes containing QS21 saponin and adsorbed to aluminum hydroxide, liposomes containing saturated phospholipids, cholesterol, and/or monophosphoryl, ALFQ, ALFA, AS01, and/or modifications or derivatives thereof.

Another embodiment of the invention is directed to immunogenic compositions comprising the peptide as disclosed herein. Preferably the immunogenic composition comprises one or more of a pharmaceutically acceptable carrier, a chemical agent, a diluent, an excipient, or an adjuvant. Preferably the pharmaceutically acceptable carrier, chemical agent, diluent, or excipient comprises water, fatty acids, lipids, polymers, carbohydrates, gelatin, solvents, saccharides, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents or a combination thereof. Preferably the adjuvant comprises alum, oil in water emulsion, amino acids, proteins, carbohydrates, Freund’s, a liposome, saponin, lipid A, squalene, liposomes adsorbed to aluminum hydroxide, liposomes containing QS21 saponin, liposomes containing QS21 saponin and adsorbed to aluminum hydroxide, liposomes containing saturated phospholipids, cholesterol, and/or monophosphoryl, ALFQ, ALFA, AS01, and/or modifications or derivatives thereof. Immunogenic compositions include vaccines.

Another embodiment of the invention is directed to antibodies that are reactive to one or more of the peptides disclosed herein. Preferably the antibody comprises IgG, IgA, IgD, IgE, IgM or fragments (e.g., Fc, Fhv, Fab) or combinations thereof. Antibodies may also be formulated into compositions for treatment of a subject. Preferably the antibody is a polyclonal, monoclonal, or partly or fully humanized antibody. Preferably the monoclonal antibody is fully or partly humanized. The monoclonal antibody may have a normal half-life or be altered to have an extended half-life. Antibodies may be included in an immunogenic composition to be administered to subjects. Another embodiment of the invention is directed to hybridomas that express monoclonal antibodies as disclosed herein.

Another embodiment of the invention comprises nucleic acids that encodes the peptides disclosed herein.

Another embodiment of the invention is directed to methods of treatment comprising administering an immunogenic composition to a subject infected or at risk of being infected by Mycobacteria. Alternatively, or in addition to the immunogenic composition, such subjects may be administered a composition comprising antibodies or monoclonal antibodies as described and discussed in this disclosure. Preferably the subject is a mammal that, after administration, generates an immune response against gram-positive bacteria and Mycobacteria. Preferably the immune response comprises opsonization, phagocytosis and/or killing of gram-positive bacteria and Mycobacteria. Also preferably, the immune response comprises generation of memory T cells against gram-positive bacteria and Mycobacteria. Preferably the gram-positive bacteria include, but not limited to Staphylococci bacteria. Preferably the Mycobacteria comprises Mycobacterium tuberculosis, Mycobacterium leprae, Mycobacterium bovis, Mycobacterium avium, and/or Mycobacterium smegmatis.

Preferably, a contiguous peptide sequence as disclosed herein includes epitopes of a bacterium and a virus which includes one or more of the sequences selected from the group of sequences consisting of SEQ ID NOs. 1-41. Also preferably, a contiguous peptide sequence comprising an epitope of a first bacterium and an epitope of a second bacterium, wherein the first bacterium and the second bacterium are of different serotypes, species or genera, which includes one or more of the sequences selected from the group of sequences consisting of SEQ ID NOs. 1-24. Also preferably, a contiguous peptide sequence comprising an epitope of a first virus and an epitope of a second virus, wherein the first virus and the second virus are of different serotypes, species or genera, which includes one or more of the sequences selected from the group of sequences consisting of SEQ ID NOs. 25-41.

Other embodiments and advantages of the invention are set forth in part in the description, which follows, and in part, may be obvious from this description, or may be learned from the practice of the invention.

### DESCRIPTION OF THE DRAWINGS

FIG. 1A Binding of MABs JG7 and GG9 hybridoma supernatant to fixed mycobacteria (strain EK-MTB, Erdman).

FIG. 1B Binding of MABs JG7 and GG9 hybridoma supernatant to fixed mycobacteria (strain HN878).

FIG. 1C Binding of MABs JG7 and GG9 hybridoma supernatant to fixed mycobacteria (strain CDC1551).

FIG. 1D Binding of MABs JG7 and GG9 hybridoma supernatant to fixed mycobacteria (strain M. smegmatis).

FIG. 2 Binding of purified MABs JG7 and GG9 to live mycobacteria.

FIG. 3A Binding of MABs JG7 and GG9 to fixed MTB - Susceptible strain H37Ra.

FIG. 3B Binding of MABs JG7 and GG9 to fixed MTB - multidrug-resistant (MDR).

FIG. 3C Binding of MABs JG7 and GG9 to fixed MTB - extensively drug-resistant (XDR) strain.

FIG. 4 Binding of MABs JG7 and GG9 to various gram-positive bacteria.

FIG. 5A Opsonophagocytic Killing Activity (OPKA) of MABs JG7 and GG9 against Mycobacterium smegmatis (SMEG) using HL-60 granulocytes.

FIG. 5B Opsonophagocytic Killing Activity (OPKA) of MABs JG7 and GG9 against Mycobacterium smegmatis (SMEG) U-937 macrophages.

FIG. 6 OPKA of MAB JG7 against Mycobacterium tuberculosis (MTB) clinical isolate STB1 using U-937 macrophages.

FIG. 7A Rapid clearance of MTB in murine blood by MAB GG9.

FIG. 7B Rapid clearance of MTB in murine blood by MAB JG7

FIG. 7C Percent mice with undetectable MAB.

FIG. 8 Binding of MABs JG7 and GG9 to Peptidoglycan (PGN).

FIG. 9 Binding profile of antisera from MS 190 immunized with PGN-CRM.

FIG. 10 Binding of anti-PGN antibodies (Day-81 sera) to fixed whole bacteria: staphylococci and mycobacteria.

FIG. 11 OPKA of Anti-PGN antibodies (Day-81 pooled sera from MS 190 group) against SMEG using the macrophage cell line U-937.

FIG. 12 Binding of Anti-PGN Hybridoma MD11 positive clones, in 24-wells, to ultrapure PGN and to various fixed gram-positive bacteria.

FIG. 13 Binding of purified anti-PGN MAB MD11 to ultrapure peptidoglycan from S. aureus and to various fixed whole bacteria.

FIG. 14 Titration of MAB MD11 binding activity to ultrapure PGN and fixed M. smegmatis.

FIG. 15A Binding of MAB JG7 to PGN peptides, PGN Pep1 - Pep6.

FIG. 15B Binding of MAB GG9 to PGN peptides, PGN Pep1 - Pep6.

FIG. 15C Binding of MAB MD11 to PGN peptides, PGN Pep1 - Pep6.

FIG. 16 Binding of MABs JG7 and MD11 to Ultrapure PGN from S. aureus.

FIG. 17 Binding of MABs LD7 and CA6 hybridoma supernatant to alpha crystallin HSP.

FIG. 18 Binding of purified MABs LD7 and CA6 to live mycobacteria.

FIG. 19 Binding of MABs LD7 and CA6 (purified from subclones) to live mycobacteria.

FIG. 20 Opsonophagocytic Killing Activity (OPKA) of MABs LD7 and CA6 against Mycobacterium smegmatis (SMEG) using U-937 macrophages.

FIG. 21A Serum antibody responses in mice immunized subcutaneously with 20 µg dose of Coronavirus Pep02. Profile of IgG1 antisera titers to the immunogens are shown as Mean ± SD.

FIG. 21B Serum antibody responses in mice immunized subcutaneously with 20 µg dose of Coronavirus Pep05. Profile of IgG1 antisera titers to the immunogens are shown as Mean ± SD.

FIG. 21C Serum antibody responses in mice immunized subcutaneously with 20 µg dose of Coronavirus Pep11. Profile of IgG1 antisera titers to the immunogens are shown as Mean ± SD.

FIG. 22A Serum antibody responses in mice immunized subcutaneously with 20 µg dose of Coronavirus Pep02. Profile of IgG2b antisera titers to the immunogens are shown as Mean ± SD.

FIG. 22B Serum antibody responses in mice immunized subcutaneously with 20 µg dose of Coronavirus Pep05. Profile of IgG2b antisera titers to the immunogens are shown as Mean ± SD.

FIG. 22C Serum antibody responses in mice immunized subcutaneously with 20 µg dose of Coronavirus Pep11. Profile of IgG2b antisera titers to the immunogens are shown as Mean ± SD.

FIG. 23A Serum antibody responses in mice immunized subcutaneously with 20 µg dose of Coronavirus Pep11 with IgG1 antisera titers to the composite coronavirus peptides.

FIG. 23B Serum antibody responses in mice immunized subcutaneously with 20 µg dose of Coronavirus Pep11 with IgG1 antisera titers to influenza epitopes.

FIG. 23C Serum antibody responses in mice immunized subcutaneously with 20 µg dose of Coronavirus Pep11 with IgG1 antisera titers o individual coronavirus spike protein and RNA polymerase epitopes.

FIG. 23D Serum antibody responses in mice immunized subcutaneously with 20 µg dose of Coronavirus Pep05 with IgG1 antisera titers to the composite coronavirus peptides.

FIG. 23E Serum antibody responses in mice immunized subcutaneously with 20 µg dose of Coronavirus Pep05 with IgG1 antisera titers to influenza epitopes.

FIG. 23F Serum antibody responses in mice immunized subcutaneously with 20 µg dose of Coronavirus Pep05 with IgG1 antisera titers to individual coronavirus spike protein and RNA polymerase epitopes.

FIG. 24A Serum antibody responses in mice immunized subcutaneously with 20 µg dose of Coronavirus Pep11. IgG2b antisera titers to the composite coronavirus peptides.

FIG. 24B Serum antibody responses in mice immunized subcutaneously with 20 µg dose of Coronavirus Pep11. IgG2b antisera titers to influenza epitopes.

FIG. 24C Serum antibody responses in mice immunized subcutaneously with 20 µg dose of Coronavirus Pep11. IgG2b antisera titers to individual coronavirus spike protein and RNA polymerase epitopes.

FIG. 24D Serum antibody responses in mice immunized subcutaneously with 20 µg dose of Coronavirus Pep05. IgG2b antisera titers to the composite coronavirus peptides.

FIG. 24E Serum antibody responses in mice immunized subcutaneously with 20 µg dose of Coronavirus Pep05. IgG2b antisera titers to influenza epitopes.

FIG. 24F Serum antibody responses in mice immunized subcutaneously with 20 µg dose of Coronavirus Pep05. IgG2b antisera titers to individual coronavirus spike protein and RNA polymerase epitopes.

FIG. 25A Serum antibody responses in select mice immunized subcutaneously with 20 µg dose of either Coronavirus Pep11 or Coronavirus Pep05. One year post primary immunizations, the selected mice were given a boost and bled a week after. IgG1 antibody titers to coronavirus peptides.

FIG. 25B Serum antibody responses in select mice immunized subcutaneously with 20 µg dose of either Coronavirus Pep11 or Coronavirus Pep05. One year post primary immunizations, the selected mice were given a boost and bled a week after. IgG1 antibody titers to influenza epitopes.

FIG. 26A Serum antibody responses in select mice immunized subcutaneously with 20 µg dose of either Coronavirus Pep11 or Coronavirus Pep05. One year post primary immunizations, the selected mice were given a boost and bled a week after. IgG antibody titers to influenza virus A.

FIG. 26B Serum antibody responses in select mice immunized subcutaneously with 20 µg dose of either Coronavirus Pep11 or Coronavirus Pep05. One year post primary immunizations, the selected mice were given a boost and bled a week after. IgG antibody titers to human Coronavirus.

FIG. 27 Neutralizing titers in select mice immunized subcutaneously with 20 µg dose of either Coronavirus Pep11 or Coronavirus Pep05. One year post primary immunizations, the selected mice were given a boost and bled a week after. Neutralization of influenza A/Hong Kong (H3N2) (ID₇₅ values).

FIG. 28A Serum antibody responses in mice immunized intradermally with 1 µg dose of a composite vaccine comprising of Coronavirus Pep11 and Coronavirus Pep05 with IgG1 antisera titers to the coronavirus peptides for each dose group.

FIG. 28B Serum antibody responses in mice immunized intradermally with 10pg dose of a composite vaccine comprising of Coronavirus Pep11 and Coronavirus Pep05 with IgG1 antisera titers to the coronavirus peptides for each dose group.

FIG. 28C Serum antibody responses in mice immunized intradermally with 20 µg dose of a composite vaccine comprising of Coronavirus Pep11 and Coronavirus Pep05 with IgG1 antisera titers to the coronavirus peptides for each dose group.

FIG. 28D Serum antibody responses in mice immunized intradermally with 1 µg dose of a composite vaccine comprising of Coronavirus Pep11 and Coronavirus Pep05. IgG1 antisera titers to influenza epitopes and universal T cell epitopes for each dose group.

FIG. 28E Serum antibody responses in mice immunized intradermally with 10pg dose of a composite vaccine comprising of Coronavirus Pep11 and Coronavirus Pep05 with IgG1 antisera titers to influenza epitopes and universal T cell epitopes for each dose group.

FIG. 28F Serum antibody responses in mice immunized intradermally with 20 µg dose of a composite vaccine comprising of Coronavirus Pep11 and Coronavirus Pep05 with IgG1 antisera titers to influenza epitopes and universal T cell epitopes for each dose group.

FIG. 29A Serum antibody responses in mice immunized intradermally with 1 µg, 10pg or 20 µg dose of a composite vaccine comprising of Coronavirus Pep11 and Coronavirus Pep05. One year post primary immunizations, the mice were given a boost and bled a week after. IgG antibody titers to influenza virus A.

FIG. 29B Serum antibody responses in mice immunized intradermally with 1 µg, 10pg or 20 µg dose of a composite vaccine comprising of Coronavirus Pep11 and Coronavirus Pep05. One year post primary immunizations, the mice were given a boost and bled a week after. IgG antibody titers to human Coronavirus.

FIG. 30 Neutralizing titers in mice immunized intradermally with 1 µg, 10 µg or 20 µg dose of a composite vaccine comprising of Coronavirus Pep11 and Coronavirus Pep05. Neutralization of influenza A/Hong Kong (H3N2).

FIG. 31A Serum antibody responses in select mice immunized intradermally with 10pg of a composite vaccine comprising of Coronavirus Pep11 and Coronavirus Pep05. One year post primary immunizations, the mice were given a boost and bled a week after with IgG1 antibody titers to coronavirus peptides.

FIG. 31B Serum antibody responses in select mice immunized intradermally with 10pg of a composite vaccine comprising of Coronavirus Pep11 and Coronavirus Pep05. One year post primary immunizations, the mice were given a boost and bled a week after with IgG1 antibody titers to influenza epitopes.

FIG. 32A Serum antibody responses in select mice immunized intradermally with 10pg of a composite vaccine comprising of Coronavirus Pep11 and Coronavirus Pep05. One year post primary immunizations, the mice were given a boost and bled a week after with IgG titers to influenza virus A.

FIG. 32B Serum antibody responses in select mice immunized intradermally with 10pg of a composite vaccine comprising of Coronavirus Pep11 and Coronavirus Pep05. One year post primary immunizations, the mice were given a boost and bled a week after with IgG titers to human Coronavirus.

FIG. 33 Neutralizing titers in select mice immunized intradermally with 10 µg of a composite vaccine comprising of Coronavirus Pep11 and Coronavirus Pep05. Neutralization of influenza A/Hong Kong (H3N2) (ID₇₅ values).

### DESCRIPTION OF THE INVENTION

Approximately one third of the world population is infected with Mycobacterium tuberculosis (MTB). Current treatment includes a long course of antibiotics and often requires quarantining of the patient. Resistance is common in many bacteria and viruses and an ever-increasing problem, as is the ability to maintain the quarantine of infected patients. Present vaccines include BCG which is prepared from a strain of attenuated (virulence-reduced) live bovine tuberculosis bacillus, Mycobacterium bovis, and live non-MTB organisms. BCG carries substantial associated risks, especially in immune compromised individuals, and has proved to be only modestly effective and for limited periods. It is generally believed that a humoral response to infection by MTB is ineffective and optimal control of infection must involve activation of T cells and macrophages.

It has been surprisingly discovered that certain regions of Mycobacterial proteins generate an immune response against Mycobacteria in mammals that can be useful in treatment or protective against infection. Proteins which contain these regions include peptidoglycan, mycolic acid, LTA, LAM, heat shock proteins, a surface antigen, a composite peptide, which may contain a composite epitope, a mimotope, a fusion peptide, a peptide conjugate, or synthetic peptide sequence. Regions of peptides that generate an immune response are antigenic regions or epitopes and peptides may contain one or more epitopes. A surface antigen is a protein that contains one or more epitopes within or outside of the membrane of a microbe or otherwise exposed or becomes exposed after a treatment on the microbe. A composite peptide is a peptide sequence that contains two or more epitopes which may be similar or dissimilar from the same of different microbes. A composite epitope is a single epitope that combines two similar epitopes creating a unique sequence and is similarly immunogenically reactive as both similar epitopes. A mimotope is an antigenic structure that possesses the same antigenic profile of a peptide or one or more epitopes, but contains a different sequence from the peptide or epitope. A fusion peptide is a peptide that comprises one or more epitopes whose construction involves enzymatic fusion or ligation. A peptide conjugate is a peptide that is chemically conjugated to another molecule that may be a peptide or a polysaccharide. A synthetic peptide is any peptide disclosed here that is chemically or otherwise synthetically manufactured.

These peptides may be obtained or copied from many different strains and/or serotypes of gram-positive bacteria, including but not limited to a Staphylococcus spp. such as Staphylococcus aureus, or a Mycobacteria spp. such as Mycobacterium tuberculosis, Mycobacterium leprae, Mycobacterium bovis, Mycobacterium avium, or Mycobacterium smegmatis. Peptides as disclosed herein can be incorporated into immunogenic composition and vaccines for the treatment of gram-positive bacterial infections including, but not limited to Staphylococcal and/or Mycobacterial infections. Immunogenic composition, vaccines, and antibodies that are reactive against the peptides can each be used to treat a Mycobacterial infection. Short-term or long-term prevention or protection from infection can be achieved with immunogenic compositions and vaccines, although often times the subject has an existing infection that requires more immediate treatment. In such instances, treatment can be administered with peptide and/or antibodies that are reactive to peptides as disclosed herein. The antibodies function immediately to clear and kill gram-positive bacteria and Mycobacteria from the blood and the peptides can induce an immune response that provides short-term or long-term protection from repeat infection.

One embodiment of the invention comprises one or more portions of gram-positive bacterial proteins and Mycobacterial proteins which include portions of peptidoglycan, mycolic acid, LTA, LAM, heat shock proteins, or a surface antigen, including a composite peptide, which may contain a composite epitope, mimotope, a fusion peptide, a peptide conjugate, and a synthetic peptide sequence thereof, and immunogenic compositions containing peptides as disclosed herein. Peptides may be from a single organism or composites of different sequences from multiple microbes to include, but not limited to viruses such as influenza virus, gram positive bacteria such as Staphylococcus or Mycobacteria (acid fast) or gram-negative bacteria. Composites can include a peptide as disclosed herein plus a carrier protein.

Preferably, the immunogenic peptide comprised of a contiguous sequence of any one of the sequences of SEQ ID NOs 1-4, 18-24, or a combination thereof. The contiguous sequence may further include one of more of the sequences selected from the group consisting of the sequences of SEQ ID NOs 5-17 and 25-41. Also preferably, the immunogenic peptide comprised of a contiguous sequence of any one of the sequences of SEQ ID NOs 25, 30, 32, 36, 38, 39, 41, or a combination thereof. The contiguous sequence may further include one or more of the sequences selected from the group consisting of the sequences of SEQ ID NOs 1-24, 26-29, 31, 33-35, 37, and 40.

Preferably the peptide contains a sequence of a viral antigen, a bacterial antigen, a parasitic antigen, a composite antigen, or a combination thereof. Also preferably, the bacterial antigen comprises an antigen of a gram-positive microorganism, a gram-negative microorganism, both gram-positive and gram-negative microorganisms, or acid-fast microorganism and may contain the sequence of a T-cell stimulating epitope, a composite epitope. Also preferably, the composite epitope comprises a bacterial or viral epitope.

Peptides of this disclosure may be coupled with carrier proteins. Preferred carrier proteins include, for example, native or recombinant cross-reactive material (CRM) or a domain of CRM, CRM197, tetanus toxin, tetanus toxin heavy chain proteins, diphtheria toxoid, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and fragments, derivatives, and modifications thereof. These peptides can be used for the treatment or prevention of a microbial infection. Peptide portions may be included in immunogenic composition which may further comprise one or more pharmaceutically acceptable carriers, chemical agents, diluents, excipients, or adjuvants. Preferably the pharmaceutically acceptable carrier, chemical agent, diluent, or excipient comprises water, fatty acids, lipids, polymers, carbohydrates, gelatin, solvents, saccharides, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents or a combination thereof. Preferred carriers include components designated as generally recognized as safe (GRAS) by the U.S. Food and Drug Administration or another appropriate authority. Preferably the adjuvant comprises alum, oil in water emulsion, amino acids, proteins, carbohydrates, Freund’s, a liposome, saponin, lipid A, squalene, liposomes adsorbed to aluminum hydroxide, liposomes containing QS21 saponin, liposomes containing QS21 saponin and adsorbed to aluminum hydroxide, liposomes containing saturated phospholipids, cholesterol, and/or monophosphoryl, ALFQ, ALFA, AS01, and/or modifications or derivatives thereof. Immunogenic compositions also include vaccines.

Another embodiment of the invention comprises antibodies that are reactive against a peptide disclosed herein. Preferred are antibodies that are reactive against peptides of gram-positive bacteria such as Staphylococcus and/or Mycobacteria. Preferably the antibody comprises IgG, IgA, IgD, IgE, IgM or fragments (e.g., Fhv, Fc, Fab, etc.) or combinations thereof. Preferably the antibody is a polyclonal, monoclonal, or humanized antibody, or an Fc portion or variable or hypervariable portion of an antibody molecule. Antibodies may be produced through recombinant techniques, such as humanization of murine antibodies preferably including a pharmaceutically acceptable carrier. Preferably the monoclonal antibody is fully or partly humanized. Preferred monoclonal antibodies include but are not limited to monoclonals identified herein as LD7, CA6, JG7, GG9, and MD11 (see U.S. Pat. No. 9,821,047 issued Nov. 21, 2017 and entitled “Enhancing Immunity to Tuberculosis,” which is incorporated by reference, and identifies JG7 as produced by hybridoma ATCC Deposit No. PTA-124416, GG9 as produced by hybridoma ATCC Deposit No. PTA-124417, and AB9 as produced by hybridoma ATCC Deposit No. PTA-124418). Another embodiment of the invention is directed to a hybridoma that expresses monoclonal antibodies as disclosed herein.

Another embodiment of the invention is directed to methods of treatment comprising administering peptides disclosed herein, immunogenic compositions disclosed herein, and/or antibodies disclosed herein to a subject in need thereof. Administering is preferably via injection into the bloodstream of the subject and can be through other routes as appropriate (e.g., IM, SQ, ID, IP). Preferably the subject is a mammal such as a human, that, after administration, generates an immune response against Mycobacteria. Preferably the immune response comprises serum antibody titers, opsonization, phagocytosis and/or killing of gram-positive bacteria or Mycobacteria. Preferably the immune response generated results in the formation of opsonizing antibodies. Also preferably, the immune response comprises the generation of memory T cells against gram-positive bacteria or Mycobacteria. Preferably the methods comprise treating or preventing latent and/or drug-resistant Mycobacteria infections such as but not limited to MTB infections. Mammals with latent infection may otherwise appear healthy, but still retain an MTB infection that often, although not always, is infectious to others. Such methods may involve administering an immunogenic composition and antibodies reactive to peptides of this disclosure such as monoclonal antibodies to the subject. Preferably the immunogenic compositions or antibodies are administered to a patient intravenously or subcutaneously and generates a humoral response that comprises generation of antibodies specifically reactive against gram-positive bacteria or preferably Mycobacterial moieties that impede host immunity or induce antibodies that enhance host immunity.

Antibodies may be fully human or produced through recombinant techniques, such as humanization of murine antibodies preferably including a pharmaceutically acceptable carrier. Preferably the antibody is specifically reactive to a peptide as disclosed herein. Preferably the peptide comprises epitopes of one or more of the gram-positive bacterial proteins such as Mycobacterial proteins, which may be produced recombinantly, synthetically, or obtained from in vitro growth of microorganisms, or a combination thereof. Preferably the pharmaceutically acceptable carrier comprises water, oil, fatty acid, carbohydrate, lipid, cellulose, or a combination thereof. Preferably peptides and antigen targets may be conjugated to other molecules such as proteins or other moieties and delivered with adjuvants such as alum, squalene oil in water emulsion amino acids, proteins, carbohydrates and/or other adjuvants.

Another embodiment of the invention is directed to monoclonal antibodies that are specifically reactive against PGN, HSPX, or mycolic acid of drug-resistant Mycobacterial infections and preferably opsonizing antibodies. Preferably the monoclonal antibody is an IgA, IgD, IgE, IgG or IgM, or an Fc fragment or variable or hypervariable region of an antibody molecule and may be derived from most any mammal such as, for example, rabbit, guinea pig, mouse, human, fully or partly humanized, chimeric or single chain of any of the above. The DNA encoding the antibodies may be utilized in any appropriate cell line to produce the encoded MABs. Another embodiment comprises hybridoma cultures that produce the monoclonal antibodies. Another embodiment of the invention comprises non-naturally occurring polyclonal antibodies that are specifically reactive against a protein of Mycobacteria.

Nucleic acid sequences that encode portions of gram-positive bacterial proteins such as Mycobacterial proteins are preferably recombinantly produced and/or synthetically manufactured. These sequences may be developed as immunogenic compositions or vaccines against gram-positive bacteria or Mycobacteria. Also preferred are nucleic acid aptamers and peptide aptamers and other molecules that mimic the structure and/or function of the portions. Also preferred are peptide and/or nucleic acid sequences that contain or encode one or more epitopes of these peptides.

Preferably, vaccines of the disclosure provide protection to the patient for greater than about one year, more preferably greater than about two years, more preferably greater than about three years, more preferably greater than about five years, more preferably greater than about seven years, more preferably greater than about ten years, and more preferably greater than about fifteen or twenty years.

Preferably the immune response generated upon the administration of an immunogenic composition or vaccine of the disclosure is protective against gram-positive bacterial infections, MTB, multi-drug resistant and/or latent TB, or another infection and enhance and/or prime the immune system of the patient to be immunologically responsive to an infection such as by promoting recognition of the pathogen, a greater and/or more rapid immunological response to an infection, phagocytosis of the pathogen or killing of pathogen-infected cells, thereby promoting overall immune clearance of the infection, including latent TB infection and reactivation TB. Preferably, a vaccination of an infected mammal promotes the activation of a humoral and/ or cellular response of the mammalian immune system. For example, administering an immunogenic composition as disclosed herein to an infected mammal promotes the sensing of the infection and then clears the infection, including latent infection, from the mammalian system by inducing or increasing phagocytic activity. Preferably this sensing and clearance activity is effective to clear the body of both active organisms and latent or dormant organisms and thereby prevent a later resurgence of the infection.

Vaccines of the invention may contain one or multiple sequences and/or portions of proteins or peptides that are derived from the same or from different source materials or organisms. Source materials include, for example, proteins, peptides, mimotopes, toxins, cell wall components, membrane components, polymers, carbohydrates, nucleic acids including DNA and RNA, lipids, fatty acids, and combinations thereof. Immunogenic compositions and vaccines with multiple portions wherein each portion comprises a different source material are referred to herein as composite peptide antigens and may include portions derived from, for example, proteins and lipids, peptides and fatty acids, and lipids and nucleic acids. Vaccine conjugates may contain portions derived from distinct organisms, such as, for example, any combination of bacteria (e.g., MTB, Strep, Staph, Pseudomonas, Clostridium), virus (e.g., RNA or DNA viruses, influenza, HIV, RSV, Zika, poliomyelitis), fungal or mold, and parasite (e.g. malaria). These conjugates may be composed of, for example, a portion of mycolic acid of MTB coupled to serum albumin (e.g., bovine serum albumin or BSA). Exemplary conjugate vaccines include, but are not limited to composite peptide antigens of MTB, peptidoglycan, mycolic acid, or LAM with a protein such as tetanus toxin or diphtheria toxin. Exemplary conjugate vaccines also include but are not limited to conjugates of a surface protein of gram-positive bacteria such as LTA with a protein such as tetanus toxin or diphtheria toxin.

Although the peptides of the disclosure may be complete vaccines against an infection in and of themselves, it has also been discovered that the peptide vaccines of the invention enhance the immune response when administered in conjunction with other vaccines against the same or a similar infection such as, for example, BCG against a TB infection. As a secondary vaccine or adjunctive treatment in conjunction with an existing primary vaccine treatment, secondary vaccines (which may be antibodies or antigens) of the invention provide a two-punch defense against infection which is surprisingly effective to prevent or extend the period of protection available from the conventional primary vaccine. The primary vaccine (i.e., conventional vaccine) and secondary vaccines (vaccines of the invention) may be administered about simultaneously, or in staggered fashion in an order determined empirically or by one skilled in the art. Preferably the peptide vaccine is administered in advance of an attenuated or killed whole cell vaccine but may also be administered after or simultaneously (e.g., collectively as a single vaccination or as separate vaccination compositions). Preferably the peptide vaccine is administered from between about two to about thirty days in advance or after administration of the whole cell vaccine, and more preferably from between about four to about fourteen days in advance or after. Without being limited as to theory, it is currently believed that the first vaccine primes the immune system of the subject, and the second vaccine provides the boost to the immune system creating a protective immunological response in the patient.

Antibodies and antibody fragments disclosed herein can be distinguished from naturally occurring antibodies and can be isolated, identified, and characterized. In addition, these antibodies may bind to chemically or structurally altered epitopes or epitopes that become exposed after the chemical treatment. For example, natural Mycobacteria possess biological material that prevents a host immune system from immunologically seeing and recognizing certain Mycobacterial antigens such as proteins and lipids, peptides, fatty acids, polysaccharides, lipids and nucleic acids. Protein or peptide examples include but are not limited to the heat-shock proteins, peptidoglycan, mycolic acid, lipoarabinomannan (LAM) and LTA. Antibodies to one or more of these biological materials induce opsonization and/or killing of microorganisms.

Another embodiment is directed to the utilization of multiple antibodies (polyclonal, monoclonal or fractions such as Fab fragments, amino acid sequences of the variable binding antibody regions, single chains, etc.) that are combined or combined with conventional antibodies (polyclonal, monoclonal or fractions such as Fab fragments, single chains, etc.) into an antibody cocktail for the treatment and/or prevention of an infection. Combinations can include two, three, four, five or many more different antibody combinations with each directed to a different peptide sequence.

Antibodies to one or more different peptides may be monoclonal or polyclonal and may be derived from any mammal such as, for example but not limited to, mouse, rabbit, goat, pig, guinea pig, rat and preferably human. Polyclonal antibodies may be collected from the serum of infected or carrier mammals (e.g., typically human, although equine, bovine, porcine, ovine, or caprine may also be utilized) and preserved for subsequent administration to patients with existing infections. Administration of antibodies for treatment against infection, whether polyclonal or monoclonal, may be through a variety of available mechanisms including, but not limited to inhalation, ingestion, and/or subcutaneous (SQ), intravenous (IV), intraperitoneal (IP), intradermal (ID), and/or intramuscular (IM) injection, and may be administered at regular or irregular intervals, or as a bolus dose.

Monoclonal antibodies may be of one or more of the classes IgA, IgD, IgE, IgG, or IgM, containing alpha, delta, epsilon, gamma or mu heavy chains and kappa or lambda light chains, or any combination heavy and light chains including effective fractions thereof, such as, for example, single-chain antibodies, isolated variable regions, isolated Fab or Fc fragments, isolated complement determining regions (CDRs), and isolated antibody monomers. Monoclonal antibodies may be created or derived from human or non-human cells and, if non-human cells, they may be chimeric MABs or humanized. Non-human antibodies are preferably humanized by modifying the amino acid sequence of the heavy and/or light chains of peptides to be similar to human variants, or genetic manipulation or recombination of the non-coding structures from non-human to human origins. The invention further comprises recombinant plasmids and nucleic acid constructions used in creating a recombinant vector and a recombinant expression vector the expresses a peptide vaccine of the invention. The invention further comprises hybridoma cell lines created from the fusion of antibody producing cells with a human or other cell lines for the generation of monoclonal antibodies of the invention. Antibodies disclosed herein promote the cell killing mechanisms of the immune system including, but not limited to phagocytosis, apoptosis, macrophage and natural-killer cell activation, cytokine and T-cell modulation and complement-initiated cell lysis.

Another embodiment of the invention is directed to the prophylactic administration of immunogenic compositions and/or antibodies to protect health care workers who administer to TB patients and, in particular, patients with multi or extreme drug resistant MTB infections. At present, a health care professional, or most anyone, who treats or cares for a patient infected with multi-drug resistant or extreme-drug resistant TB is at extreme risk for acquiring the same infection as those he or she cares for. There is also a substantial risk to all persons within a general health care facility that such a TB infection will be acquired by other health care workers at the facility or visitor who otherwise have no contact or interaction with such patients. With the prophylactic administration of antibodies or vaccines of the invention to health care workers, they are able to care for and attend these patients. With the administration of immunogenic compositions of the invention, preferably monoclonal antibodies or vaccines, a health care worker may be protected from nosocomial and occupationally acquired TB or gram-positive bacterial infections for weeks, months and longer.

Additionally the vaccine antigens and/or antibodies of the invention may be administered in conjunction with conventional vaccines against gram-positive bacteria and MTB (e.g., BCG) or as a Prime Boost with another vaccine such as, for example BCG. This combined vaccine of the invention provides an enhancement of the immune response generated and/or extends the effectiveness and/or length of period of immunity. Enhancement is preferably an increase in the immune response to MTB infection such as an increase in the cellular or humoral response generated by the host’s immune system. An effective amount of vaccine, adjuvant and enhancing antigen of the invention is that amount which generates an infection clearing immune response or stimulates phagocytic activity. Upon administration of the combined vaccine, an increase of the cellular response may include the generation of targeted phagocytes, targeted and primed natural killer cells, and/or memory T cells that are capable of maintaining and/or promoting an effective response to infection for longer periods of time than the conventional vaccine would provide alone. An increase in the humoral response may include the generation of a more diverse variety of antibodies including, but not limited to different IgG isotypes or antibodies to more than one microbe or more than one MTB molecule that are capable of providing an effective response to prevent infection by MTB and/or another microbe as compared to the humoral response that would be generated from just a conventional MTB vaccine. Administration preferably comprises combining BCG vaccine and a vaccine antigen that generates a humoral response in the patient to a surface antigen of MTB. Preferably the response is to mycolic acid, peptidoglycan, lipoarabinomannan and/or another component of the microorganism, preferably one that presents or is otherwise exposed on the surface of MTB or secreted during infection. Some substances produced by MTB may be toxic to the host immune system or impede immune function. Antibodies that clear or neutralize these toxic substances (such as released or free mycolic acid components) can further act to enhance and improve host immunity.

Treatment of subjects may be combined with antibiotics, cytokines and other bactericidal and/or bacteriostatic substances (e.g., substances that inhibit protein or nucleic acid synthesis, substances that injury membrane or other microorganism structures, substances that inhibit synthesis of essential metabolites of the microorganism), or one or more substances that attacks the cell wall structure or synthesis of the cell wall of the microorganism. Effective amounts of antibiotics are expected to be less than the manufacture recommended amount or higher dose, but for short periods of time (e.g., about one hour, about 4 hours, about 6 hours, less than one or two day). Examples of such antibiotics include but are not limited to one or more of the chemical forms, derivatives and analogs of penicillin, amoxicillin, Augmentin (amoxicillin and clavulanate), polymyxin B, cycloserine, autolysin, bacitracin, cephalosporin, vancomycin, and beta lactam. Antibiotics work synergistically with the antigens of the invention to provide an efficient and effective preventative or treatment of an infection. The antibiotics are not needed in bacteriostatic or bactericidal quantities, which is not only advantageous with regard to expense, availability and disposal, these lower dosages do not necessarily encourage development of resistance to the same degree, together a tremendous benefit of the invention.

Antibodies may be administered directly to a patient to treat or prevent infection via inhalation, oral, SQ, IM, IP, ID, IV or another effective route, often determined by the physical location of the infection and/or the infected cells. Treatment is preferably one in which the patient does not develop or develops only reduced symptoms (e.g., reduced in severity, strength, period of time, and/or number) associated with infection and/or does not become otherwise contagious. Antibodies used alone or in conjunction with anti-Mycobacterial antibiotics will increase the clearance of organisms from the blood or other tissues, or inactivate substances that impede immunity as measured by a more rapid reduction of symptoms, more rapid time to smear negativity and improved weight gain and general health. In addition, treatment provides an effective reduction in the severity of symptoms, the generation of immunity to Mycobacteria, and/or the reduction of infective period of time. Preferably the patient is administered an effective amount of antibodies to prevent or overcome an infection alone or as adjunctive therapy with antibiotics.

Although the invention is generally described in reference to human infection by Mycobacterium tuberculosis, as is clear to those skilled in the art the compositions including many of the antibodies, tools and methodology is generally and specifically applicable to the treatment and prevention of gram-positive bacterial infections and many other diseases and infections in many other subjects (e.g., cats, dogs, pets, horses, cattle, pigs, farm animals, etc.) and most especially diseases wherein the causative agent is of viral, bacterial, fungal and parasitic origins.

The following examples illustrate embodiments of the invention but should not be viewed as limiting the scope of the invention.

### EXAMPLES

### Example 1

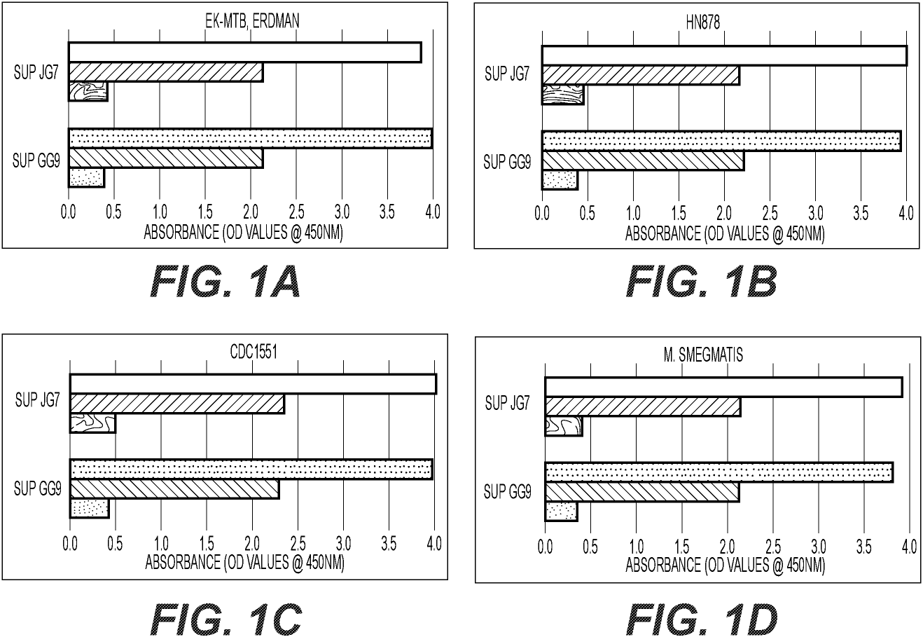

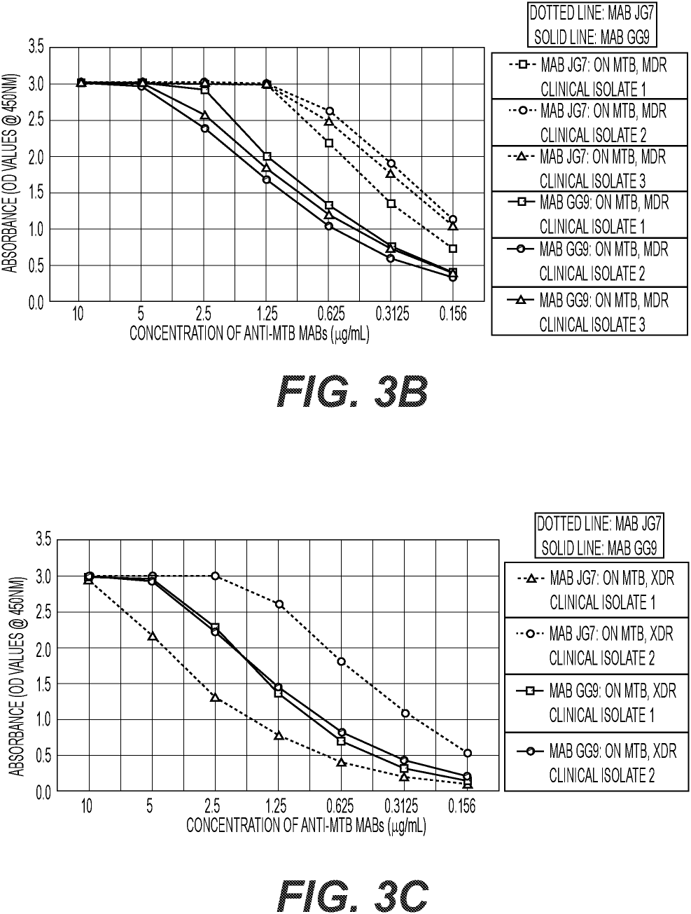

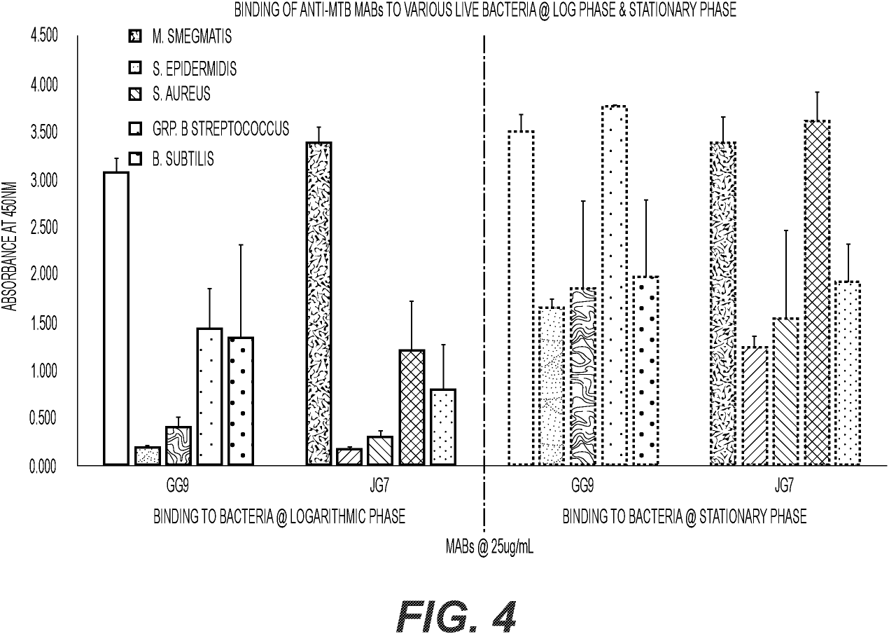

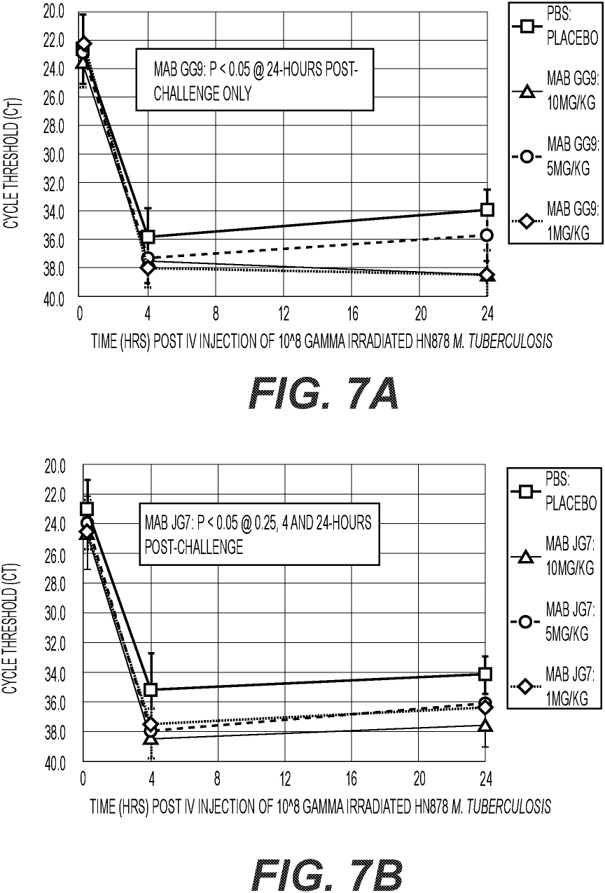

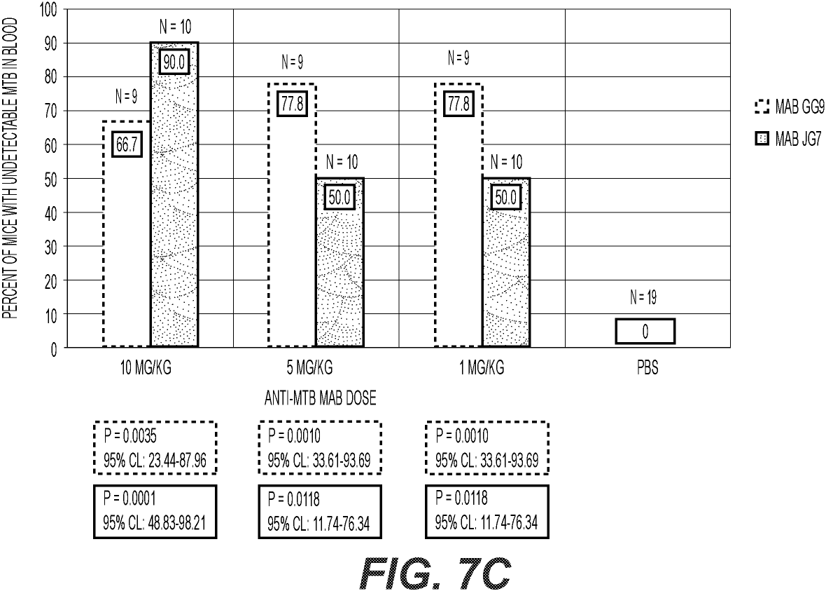

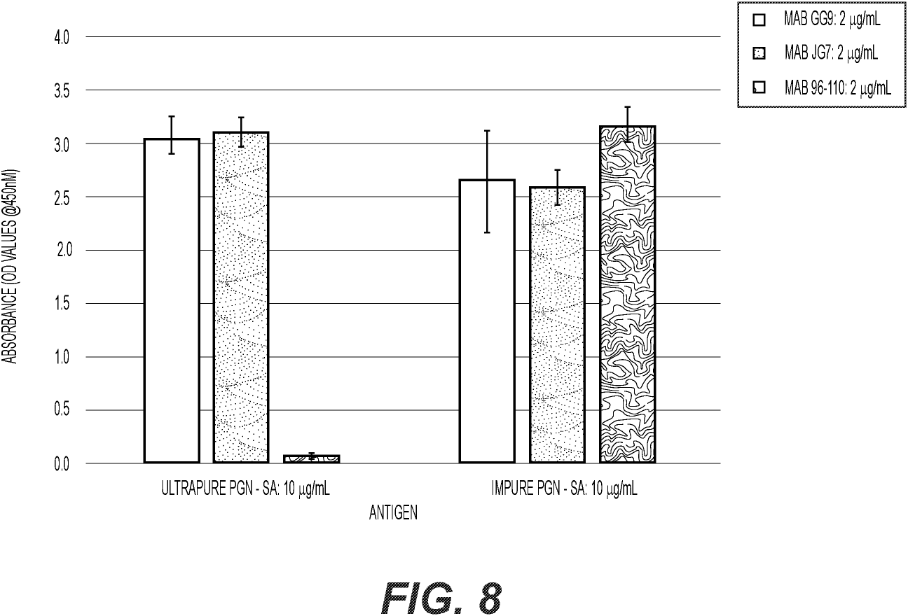

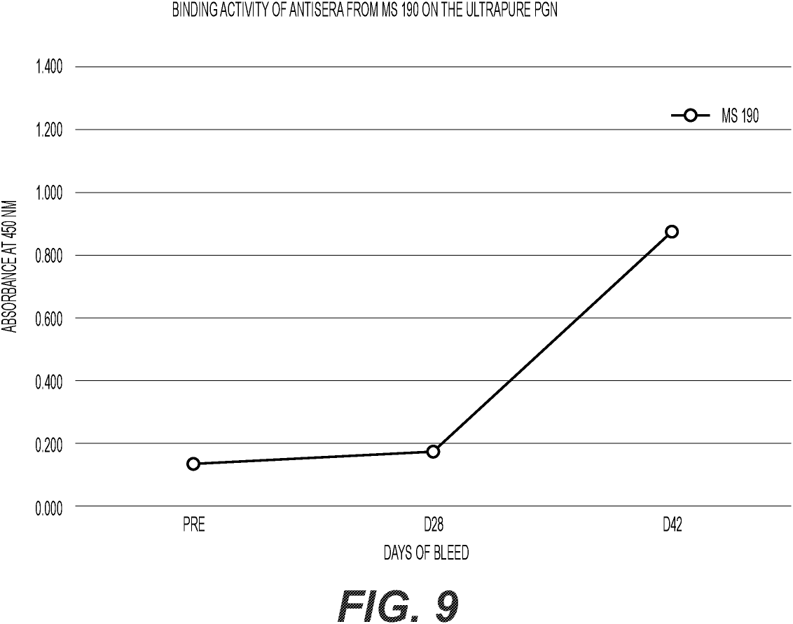

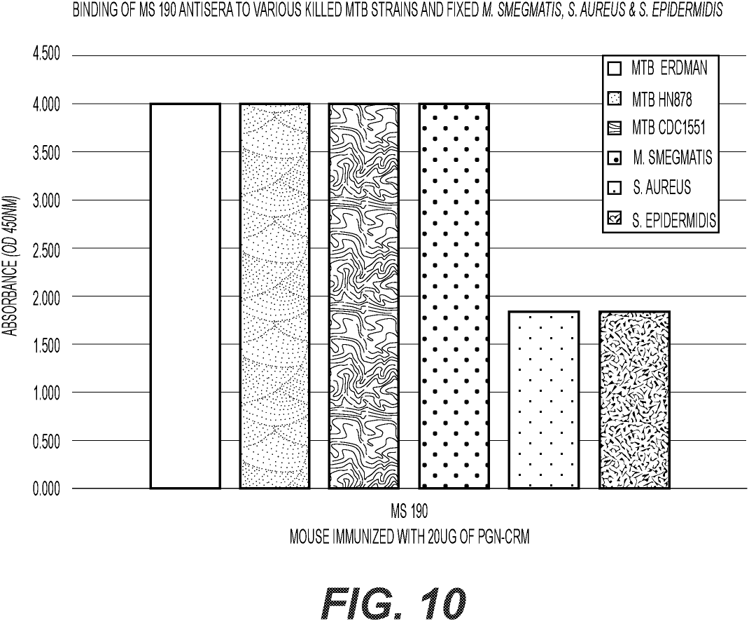

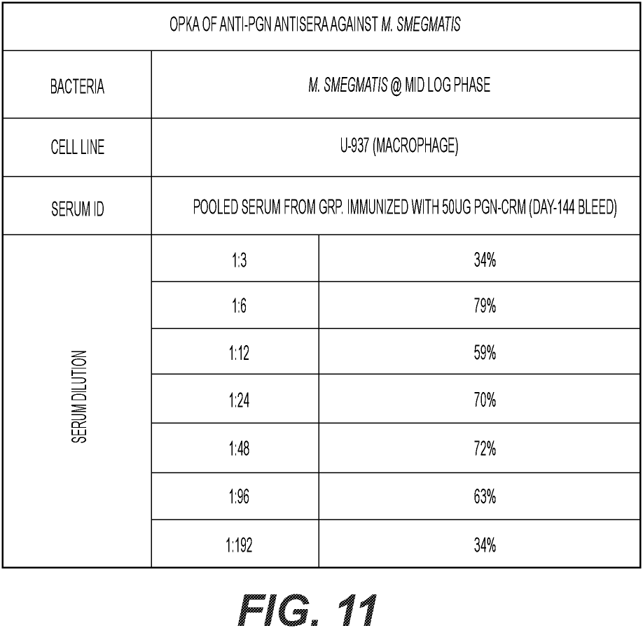

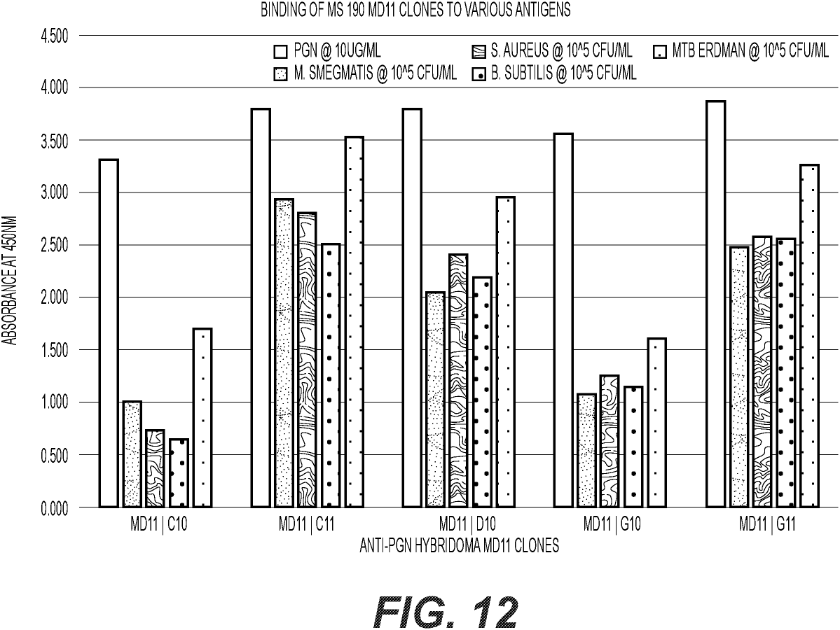

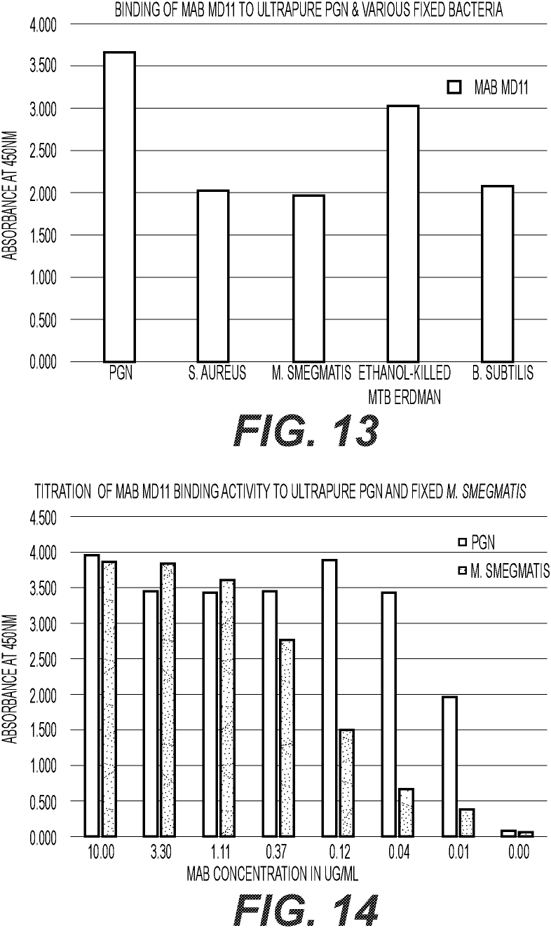

Monoclonal Antibodies (MABs) JG7, GG9, and MD11 were developed against a Mycobacterium tuberculosis (MTB) and gram-positive bacteria cell wall component peptidoglycan (PGN). Mouse splenocytes were fused with SP2/0 myeloma cells for production of hybridomas and MABs. MAB JG7 (IgG1) was derived from BALB/c MS 1323 immunized intravenously with Ethanol-killed Mycobacterium tuberculosis (EK-MTB), without adjuvant. Killing of MTB using Ethanol may have altered the MTB capsule exposing deeper cell wall epitopes. MAB GG9 (IgG1) was derived from BALB/c MS 1420 immunized subcutaneously with EK-MTB, without adjuvant. MAB MD11 (IgG2b) was derived from ICR MS 190 immunized subcutaneously with ultrapure Peptidoglycan (PGN), conjugated to CRM197 and adjuvanted with TITERMAX® Gold. EK-MTB and PGN were immunogenic in mice. Serum antibodies that bound to gram-positive bacteria and MTB and promoted opsonophagocytic killing (OPKA) of the bacteria by phagocytic effector cells. Monoclonal antibodies (MABs) JG7 and GG9 produced (from mice 1323 and 1420. respectively), bound to M. smegmatis, multiple MTB strains and susceptible. MDR, and XDR clinical isolates (FIGS. 1A, 1B, 1C, 1D, 3A, 3B, 3C). The MABs also demonstrated broad bacterial binding and enhanced OPKA against MTB and M. smegmatis (FIGS. 4, 5A, 5B, 6). In addition, the MABs promoted rapid clearance of MTB from the blood of mice given as little as 1 mg/kg (FIGS. 7A, 7B, 7C). MABs JG7 and GG9 are IgG1 and both MABs bound to ultra-pure peptidoglycan (PGN) (FIG. 8). Mice were subsequently immunized with CRM-conjugated PGN, and serum antibodies were induced that also reacted broadly across gram-positive bacteria and MTB. Moreover, the mice produced serum antibodies that bound to PGN and fixed bacteria. Mouse 190 (MS 190) with anti-PGN serum antibodies that also bound broadly to bacteria and enhanced OPKA was selected for hybridoma production (FIGS. 9 -11). MAB MD1 1 which was identified from the hybridomas that were produced is an IgG2 MAB that binds across multiple bacteria and ultra-pure PGN (FIGS. 12 and 13). Conjugated PGN immunization induced broadly reactive antibodies to bacteria.

MABs JG7 and GG9 showed binding activity to killed MTB. live Mycobacterium smegmatis (SMEG) and several strains of live MTB - susceptible. MDR and XDR. In addition. JG7 and GG9 promoted opsonophagocytic killing of SMEG and MTB using macrophage and granulocytic cell lines and enhanced clearance of MTB from blood (FIGS. 1-8).

Binding activities of supernatants from hybridomas JG7 and GG9 to Mycobacterium tuberculosis (MTB) and Mycobacterium smegmatis (SMEG), evaluated at dilutions 1:10. 1:100 and 1:1000 on fixed mycobacteria at 1x10⁵ CFU/well. FIG. 1A, FIG. 1B FIG. 1C. respectively, shows binding of supernatant to killed MTB Erdman, HN878 and CDC1551. FIG. 1D depicts binding of supernatants to fixed SMEG. OD values for growth media without antibody (negative control) range between 0.046 - 0.060,

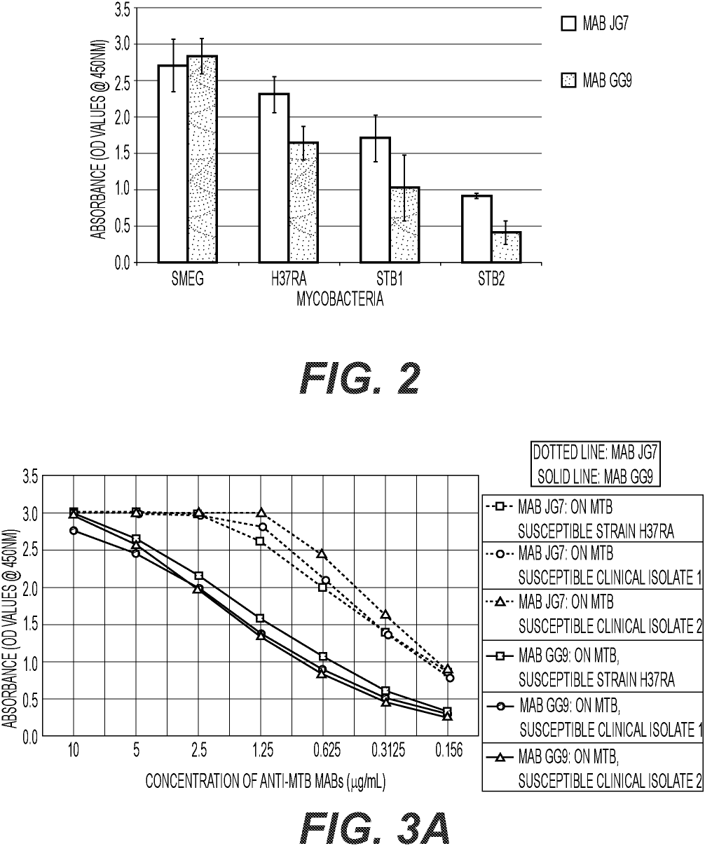

Binding activity of purified anti-Mycobacterium tuberculosis monoclonal antibodies (anti-MTB MABs) GG9 and JG7 to live Mycobacterium smegmatis (SMEG) and live susceptible MTB H37Ra (1ab strain) and STB 1 and STB2 (susceptible clinical isolates) as demonstrated in a Live Bacteria ELISA (see FIG. 2). Data (expressed as mean ± standard errors; n=3) are representative of three individual experiments.

Binding activity of purified anti-Mycobacterium tuberculosis monoclonal antibodies (anti-MTB MABs) JG7 and GG9 to fixed MTB at 1x10⁵ CFU/well. FIG. 3A demonstrates MAB binding to susceptible H37Ra strain and clinical isolates 1, and 2; FIG. 3B to multidrug-resistant (MDR) clinical isolates 1, 2 and 3; and FIG. 3C to extensively drug-resistant (XDR) clinical isolates 1 and 2. Data (expressed as mean) are representative of three individual experiments.

Binding activity of anti-MTB MABs JG7 & GG9 to various live gram-positive bacteria grown to either log phase or stationary phase as screened in the Live Bacteria ELISA (see FIG. 4).

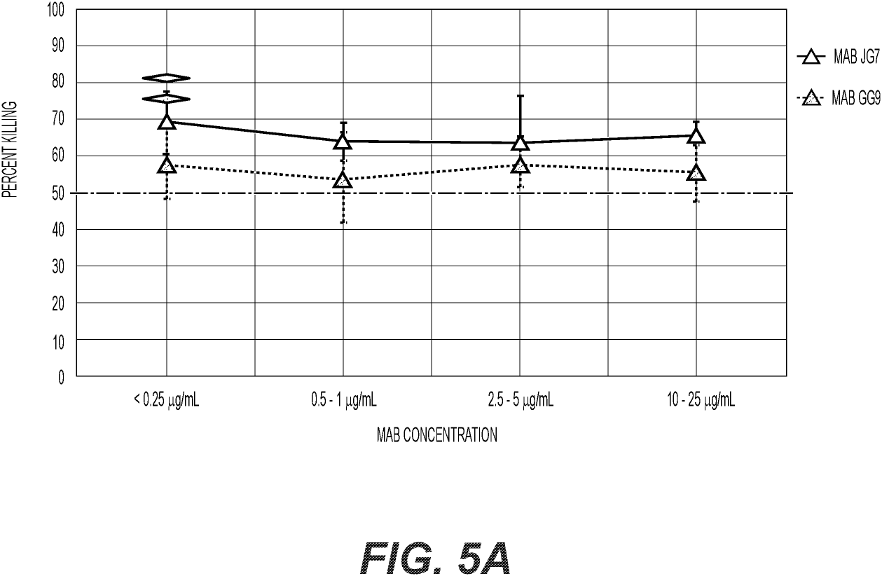

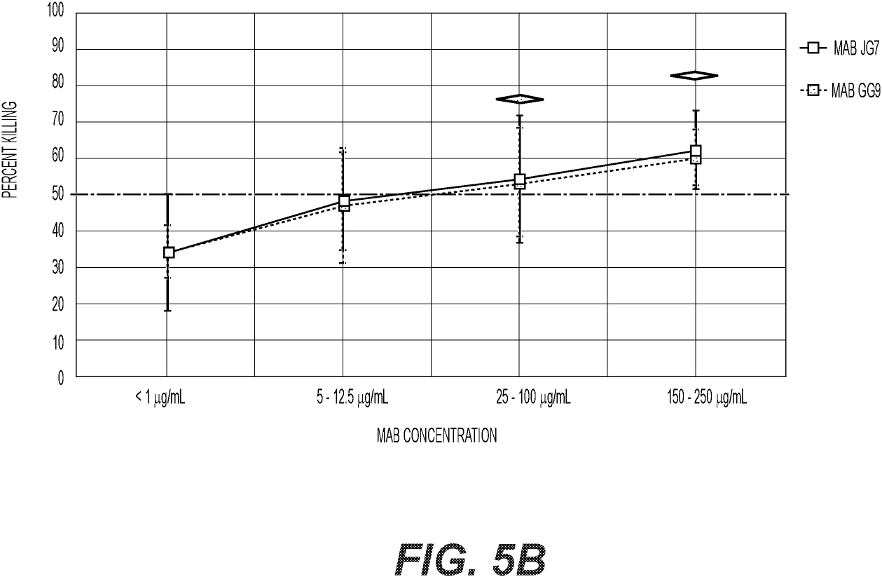

Enhanced OPKA of MABS JG7 and GG9 against Mycobacterium smegmatis (SMEG) using HL60 granulocytes and Clq (FIG. 5A) occurred at low antibody concentrations (<0.25 µg/ml) and stayed constant when antibody levels were increased over one hundred-fold. While MAB JG7 consistently had higher percent killing, the difference did not reach statistical significance. Peak OPKA for both JG7 and GG9 occurred at 0.06 µg/mL and were 81 % and 76%, respectively. In FIG. 5B, enhanced MAB OPKA against SMEG using U-937 macrophages (without C1q) was significantly more pronounced at higher antibody concentrations (JG7: p = 0.0001, GG9: p < 0.0001) and both MABs tracked closely together across all antibody concentrations. Peak OPKA for JG7 and GG9 were 82% at 175 µg/mL and 76% at 100 µg/mL), respectively.

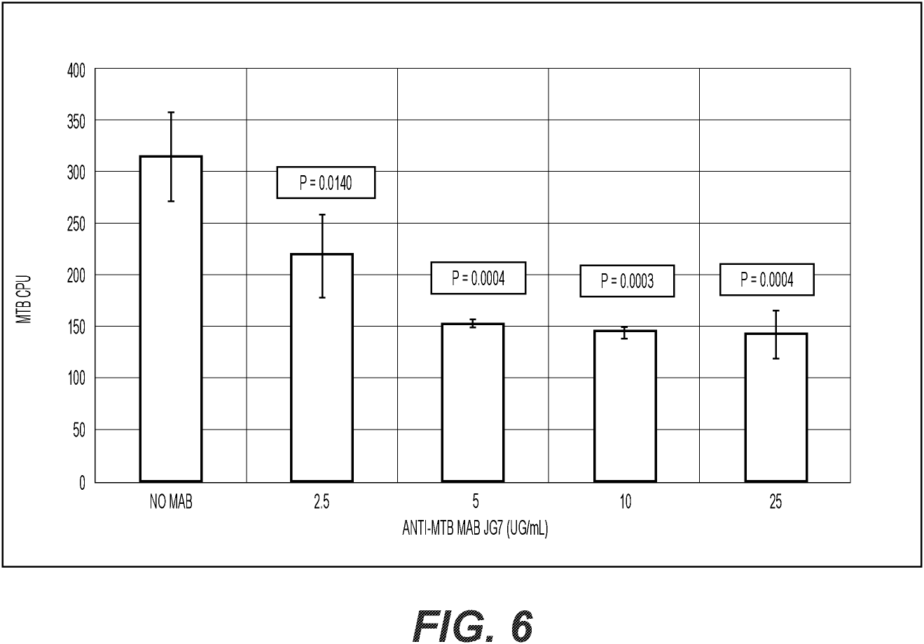

OPKA of MAB JG7 against live Mycobacterium tuberculosis (MTB) clinical isolate STB1, using U-937 macrophages (without C1q) was significantly enhanced at MAB levels 2.5 -25 µg/mL (see FIG. 6). Compared to the control sample wells (without MAB), antibody sample wells had CFU counts that were significantly reduced (p < 0.5) from 315 (No MAB) to 219 (2.5 µg/mL), 154 (5 µg/mL), 145 (10 µg/mL) and 143 (25 µg/mL).

Using qPCR, rapid clearance of Mycobacterium tuberculosis (MTB) in blood was observed in all groups from the in vivo study with N=76 ICR mice. While MAB GG9 (FIG. 7A) significantly enhanced blood clearance at 24 hours post challenge (1 mg/kg p= 0.0021, 10 mg/kg p= 0.0013), MAB JG7 (FIG. 7B) significantly enhanced clearance at all time points (0.25, 4 and 24 hours) and at one or more doses. FIG. 7C shows the percentage of mice with undetectable levels of MTB in blood according to qPCR. Statistical significance determined by comparison of MAB-treated vs. PBS-treated blood samples from mice according to no detection (i.e., C_{T}=40, qPCR) was calculated using the Chi-squared test, with significance threshold set at p < 0.05 and 95% confidence intervals shown.

MABs JG7 and GG9 and anti-LTA MAB (96-110) were analyzed for binding to a cell wall mixture and Ultrapure PGN, both from Staphylococcus aureus (FIG. 8). Compared to a control MAB 96-110 directed against LTA that only bound to impure cell wall mixture containing components including LTA and PGN, MABs JG7 and GG9 bound to both cell wall mixture and ultrapure PGN (that does not contain other cell wall components such as LTA). This strongly suggests that MABs JG7 and GG9 bind to an epitope on PGN. PGN-binding activity of MABs GG9 and JG7 was demonstrated to Ultrapure and Impure PGN, while anti-LTA MAB 96-110 only bound the Impure PGN.

MAB MD11 showed binding activity to Peptidoglycan, killed MTB, and various strains of gram-positive bacteria (see FIGS. 12 and 13). In addition, MD11 promoted opsonophagocytic killing of SMEG and Staphylococci (>50% OPKA) using macrophages (U-937 cell line) and polymorphonuclear cells (PMNs), respectively (FIG. 14).

### Example 2

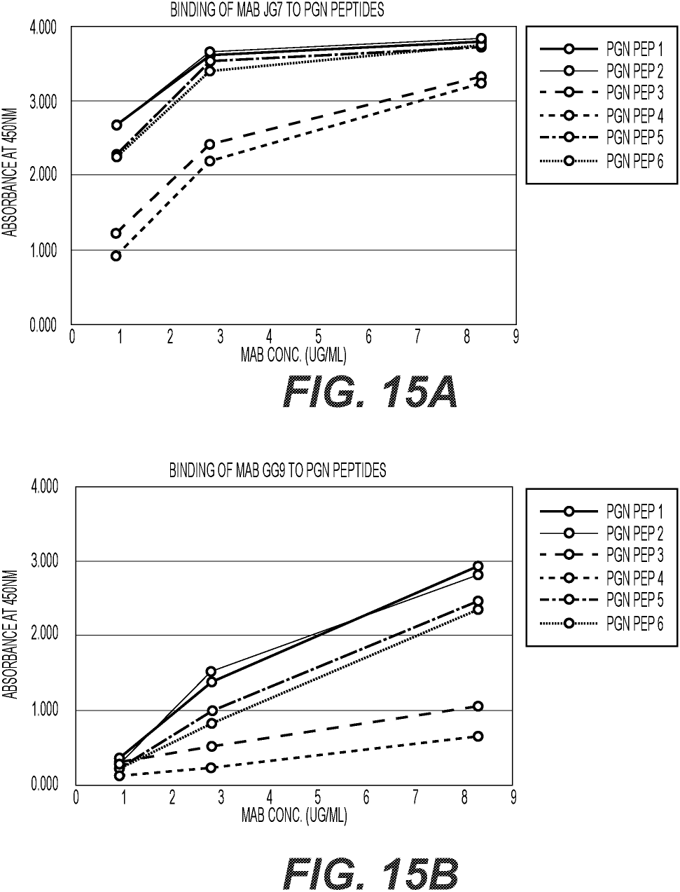

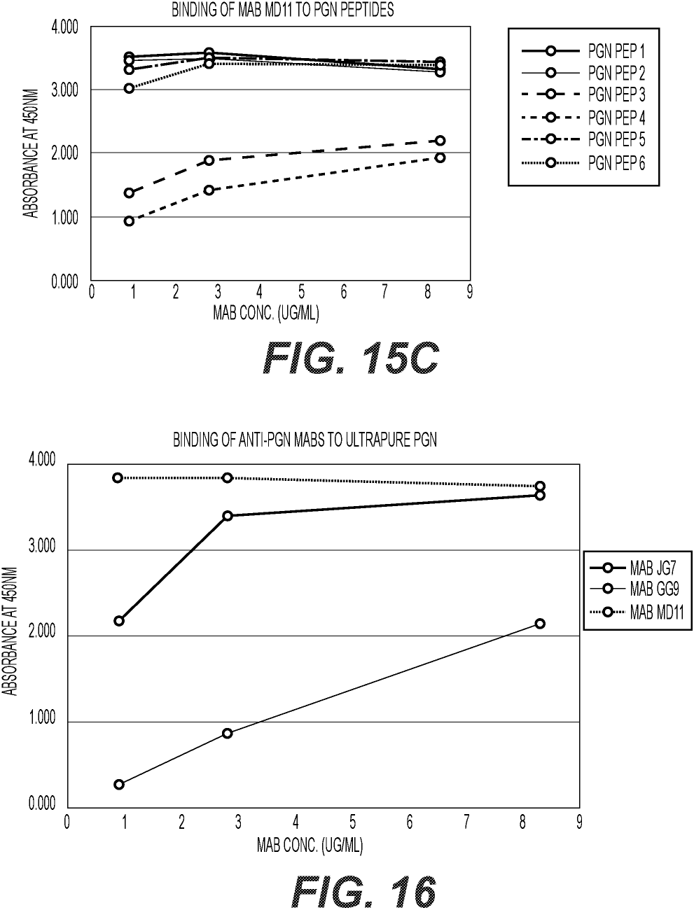

MABs JG7, GG9 and MD11 were analyzed for binding to small, synthesized peptides (see FIGS. 15A, 15B and 15C) and to ultra-pure PGN (FIG. 16). MABs JG7 and GG9 are from mice immunized with ethanol killed MTB and MAB MD11 from a mouse immunized with CRM-conjugated PGN. Each of the MABs bound to all the small individual peptides and to PGN, but the binding patterns across the peptides were different.

TABLE 1

PGN Peptide Sequences

| SEQ ID NO | Peptide number | Peptide ID | Peptide Sequence |
|---|---|---|---|
| 1 | PGN Pep01 | LVD-PSEQ-A-PGN Pep 01 | AEKAGGGGGAEKA |
| 2 | PGN Pep02 | LVD-PSEQ-A-PGN Pep 02 | AEKAEKAGGGGGAEKAEKA |
| 3 | PGN Pep03 | LVD-PSEQ-A-PGN Pep 03 | QYIKANSKFIGITEAEKAGGGGAEKA |
| 4 | PGN Pep04 | LVD-PSEQ-A-PGN Pep 04 | AEKAGGGGGAEKAQYIKANSKFIGITE |
| 5 | PGN Pep05 | LVD-PSEQ-A-PGN Pep 05 | AEKA |
| 6 | PGN Pep06 | LVD-PSEQ-A-PGN Pep 06 | AEKAGGGGG |

SEQ ID NO 7: QYIKANSKFIGITE = tetanus universal T cell epitope
SEQ ID NO. 8: GGGGG = pentaglycine bridge

### Example 3

Monoclonal antibodies (MABs) were developed against Mycobacterium tuberculosis Alpha Crystallin Heat Shock Protein. MAB LD7 (IgG2a) was derived from BALB/c MS 1435 immunized subcutaneously with TB Pep01 (Conserved Alpha Crystallin HSP), with Freund’s adjuvant. MAB CA6 (IgG2b) was derived from BALB/c MS 1435 immunized subcutaneously with TB Pep01 (Conserved Alpha Crystallin HSP), with Freund’s adjuvant.

PGN epitopes shown in Table 1 can be mixed and matched in varied combinations such as with or without a T cell epitope, to produce composite peptides and mixtures that could be formulated with adjuvants as MTB or Staph/Gram positive bacterial vaccines.

TABLE 2

MTB, LAM, and Staphylococcus LTA Peptide Sequences

| SEQ ID NO | Peptide number | Peptide ID | Peptide Sequence | Description |
|---|---|---|---|---|
| 9 | TB Pep01 | LvD-PSEQ-A-TB Pep 01 | SEFAYGSFVRTVSLPVGADE | Conserved MTB Alpha Crystallin HSP Epitope |
| 10 | TB Pep02 | LvD-PSEQ-A-TB Pep 02 | SEFAYGSFVRTVSLPVGADEGNLFIAPWGVIHHPHYEECSCY | Conserved MTB Alpha Crystallin HSP Epitope and 2 conserved influenza HA epitopes and 1 conserved NA Epitope |
| 11 | LAM Pep01 | LvD-PSEQ-A-LAM Pep 01 | HSFKWLDSPRLR | Conserved MTB Lipoarabinomanin Mimotope |
| 12 | LAM Pep02 | LvD-PSEQ-A-LAM Pep 02 | ISLTEWSMWYRH | Conserved MTB Lipoarabinomanin Mimotope |
| 13 | LTA Pep01 | LvD-PSEQ-A-LTA Pep 01 | WRMYFSHRHAHLRSP | LTA Epitope |
| 14 | LTA Pep02 | LvD-PEQ-A-LTA Pep 02 | WHWRHRIPLQLAAGR | LTA Epitope |

SEQ ID No. 15: GNLFIAPWGVIHHPHYEECSCY = composite influenza peptide comprising HA and NA epitopes
SEQ ID No. 16: SEFAYGSFMRSVTLPPGADE = M. smegmatis peptide sequence

MTB, LAM and Staphylococcus LTA epitopes shown in Table 2 are mixed and matched in combinations such as with or without a T cell epitope, to produce composite peptides and mixtures that are formulated with adjuvants as MTB or Staph/Gram positive bacterial vaccines.

MABs LD7 and CA6 showed highly specific binding to the alpha crystallin HSP (TB Pep01) and promoted opsonophagocytic killing of M. smegmatis (SMEG) (see FIGS. 16-20).

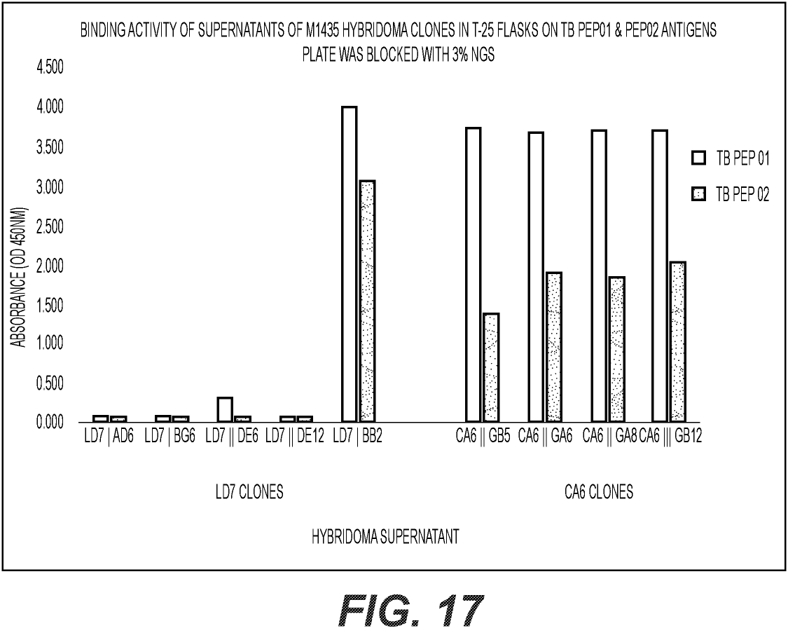

FIG. 17 depicts binding activities of supernatants from hybridomas LD7 and CA6 to TB Pep01 and TB Pep02 at 1 µg/mL. OD values (450 nM) for growth media without antibody (negative control) range between 0.046 - 0.060.

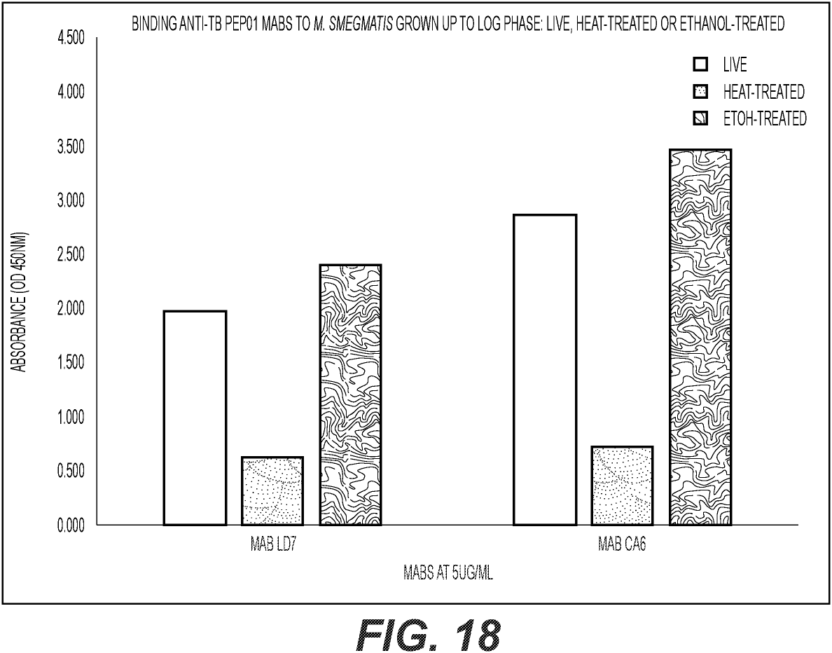

FIG. 18 depicts binding activity of purified anti-TB Pep01 MABs LD7 and CA6 to live Mycobacterium smegmatis (SMEG) as demonstrated in a live bacteria ELISA. MABs were purified from original hybridomas. There is 80% homology (16 out of 20 amino acids) of HSP20 between M. tuberculosis (SEQ ID NO 9; SEFAYGSFVRTVSLPVGADE) and M. smegmatis (SEQ ID NO 16; SEFAYGSFMRSVTLPPGADE).

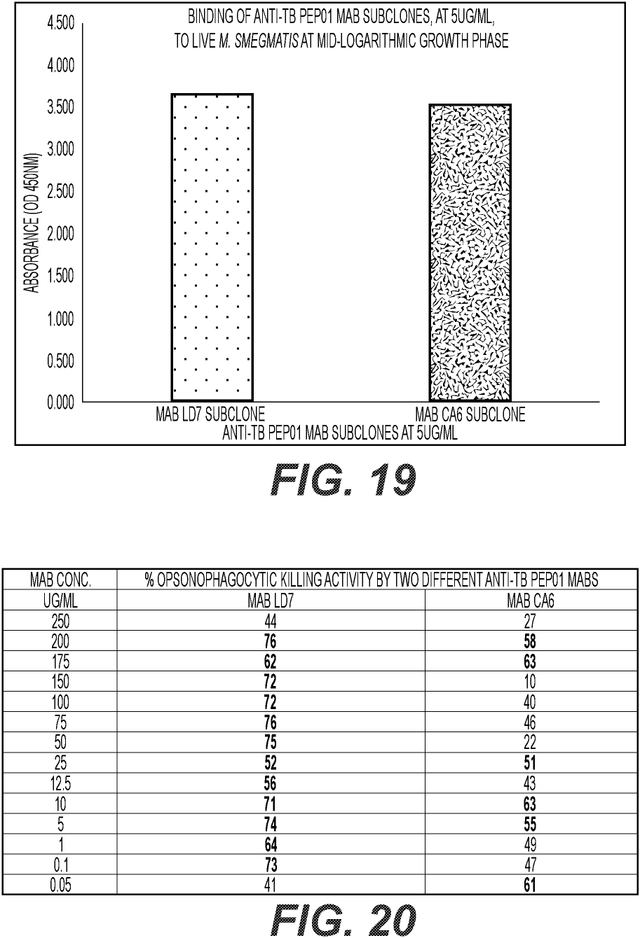

FIG. 19 depicts binding activity of purified anti-TB Pep01 MABs LD7 and CA6 to live Mycobacterium smegmatis (SMEG) as demonstrated in a Live Bacteria ELISA. MABs were purified from hybridoma subclones.

FIG. 20 depicts enhanced OPKA of MABs LD7 and CA6 against Mycobacterium smegmatis (SMEG) using U-937 macrophages. Peak OPKA for LD7 was 76% and for CA6 was 63%.

Mouse 1435 immunized with a conserved MTB alpha crystallin heat shock protein epitope developed serum antibodies that bound to a small synthesized alpha crystallin HSP peptide (TB Pep01). MAB LD7 (IgG2a) and MAB CA6 (IgG2b) that were subsequently produced from MS 1435 bound broadly to TB Pep01, TB Pep02 (composite peptide that constitutes TB Pep01, two conserved influenza hemagglutinin epitopes, and one conserved neuraminidase epitope), and M. smegmatis (FIGS. 17-19). In addition, these MABs showed enhanced OPKA (>50%) against M. smegmatis (FIG. 20).

The HSP epitope elicited strong humoral responses in mice, with high serum antibody titers and subsequently generated two MABs - LD7 and CA6 (IgG2a and IgG2b isotypes, respectively). These MABs bound strongly to the HSP epitope (OD450nm of 3.0-3.5) but had low binding activity to fixed mycobacteria (OD450nm < 0.25). Notably, MABs LD7 and CA6 showed significantly increased binding activity to live SMEG, compared to fixed SMEG, and surprisingly demonstrated significant OPKA against SMEG at both low (0.1 µg/mL) and high (200 µg/mL) antibody concentrations.

The small conserved synthetic HSP epitope induced a robust humoral response in mice and generated two MABs that recognized live SMEG and demonstrated significant OPKA against SMEG at MAB concentrations as low as 0.1 µg/mL. Immunization with this small conserved synthetic HSP epitope generates opsonic antibody responses against mycobacteria and provide important strategies for TB vaccines and therapeutics.

### Example 4

### Composite Peptide TB, Gram Positive Bacteria and Influenza/Coronavirus Vaccines

The 16.3 KD alpha crystallin heat shock protein (HSP16.3) belongs to the small heat shock protein (HSP20) family. It plays a major role for MTB survival, growth, virulence, and cell wall thickening. TB Pep 01 is a highly conserved region of HSP16.3 and immunization of mice induced antibodies that bind to mycobacteria and promote opsonophagocytic killing of M. smegmatis (see Example 3). Peptidoglycan is a cell wall component that is common across many bacteria and antibodies to PGN bind to MTB (and other gram-positive bacteria). Immunization of mice with ethanol killed MTB induced anti-PGN antibodies that promoted phagocytic killing of MTB. In addition, these antibodies bind to small PGN epitopes and composite antigens (Table 1). Cell wall PGN composite peptides and HSP16.3 the highly conserved peptide (TB Pep 01) are mixed and matched to produce composite peptides and mixtures with or without an added T cell epitope to provide vaccines to produce broadly protective immunity across large groups of bacteria (Table 3). In addition, combining HSP16.3 with PGN epitopes provides a TB vaccine that targets active MTB infection and latency. This vaccine is used alone or in combination with BCG as a booster vaccine with BCG, or other TB vaccines. In a similar fashion, LTA mimotopes combined with PGN epitopes provide an example of a broad composite peptide gram positive bacterial vaccine, while mixing coronavirus and influenza peptides provides a prototype composite peptide vaccine for prevention or treatment of infections by these viruses. (Table 3)

TABLE 3

MTB, PGN, and Other Microbial Peptides and Composite Peptide Antigens

| SEQ ID NO | Peptide number | Peptide ID | Peptide Sequence |
|---|---|---|---|
| 17 | TB Pep01 | LVD-PSEQ-A-TB Pep 01 | SEFAYGSFVRTVSLPVGADE |
| 18 | PGN.TB Pep01 | LVD-PSEQ-A-PGN.TB Pep01 | AEKAGGGGGAEKASEFAYGSFVRTVSLPVGADE |
| 19 | PGN.TB Pep02 | LD-PSEQ-A-PGN.TB Pep02 | AEKAGGGGGAEKASEFAYGSFVRTVSLPVGADEQYIKANSKFIGITE |
| 20 | PGN.TB Pep03 | LD-PSEQ-A-PGN.TB Pep03 | SEFAYGSFVRTVSLPVGADEAEKAGGGGGAEKA |
| 21 | PGN.TB Pep04 | LD-PSEQ-A-PGN.TB Pep04 | AEKAGGGGGSEFAYGSFVRTVSLPVGADEGGGGGAEKA |
| 22 | PGN.TB Pep05 | LVD-PSEQ-A-PGN.TB Pep05 | AEKAGGGGGSEFAYGSFVRTVSLPVGADEGGGGGAEKAQYIKANSKFIGITE |
| 23 | PGN.LT A Pep01 | LVD-PSEQ-A-PGN.LTA PepOl | WRMYFSHRHAHLRSPGGGGGAEKAGGGGGQYIKANSKFIGITE |
| 24 | PGN.LT A Pep02 | LVD-PSEQ-A-PGN.LTA Pep02 | WHWRHRIPLQLAGRAEKAGGGGGWRMYFSHRHAHLRSPQYIKANSKFIGITE |
| 25 | Coronavirus Pep05 | LVD-PSEQ-A-Coronavirus Pep06 | WDYPKCDRATEVETPIRNEHYEECSCYQYIKANSKFIGITE |
| 26 | Flu Pep03 | LVD-PSEQ-A-Flu Pep03 | GNLFIAP |
| 27 | Flu Pep06 | LVD-PSEQ-A-Flu Pep06 | WGVIHHP |
| 28 | Flu Pep 10 | LVD-PSEQ-A-Flu Pep10 | HYEECSCY |
| 29 | Coronavirus Pep13 | LVD-PSEQ-A-Coronavirus Pep 13 | YFPLQSYGFQPTNGVGYQPYR |
| 30 | Coronavirus Pep14 | LVD-PSEQ-A-Coronavirus Pep 14 | YFPLQSYGFQPTNGVGYQPYRQYIKANSKFIGITE |
| 31 | Coronavirus Pep15 | LVD-PSEQ-A-Coronavirus Pep15 | YQAGSTPCNGVEGFNCYFPLQ |
| 32 | Coronavirus Pep16 | LVD-PSEQ-A-Coronavirus Pep16 | YQAGSTPCNGVEGFNCYFPLQYIKANSKFIGITE |
| 33 | Flu Pep52 | LVD-PSEQ-A-Flu Pep52 | ETPIRNE |
| 34 | Flu Pep53 | LVD-PSEQ-A-Flu Pep53 | TEVETPIRNE |
| 35 | Flu Pep57 | LVD-PSEQ-A-Flu Pep57 | SLLTEVETPIRNEWGLLTEVETPIR |
| 36 | Coronavirus Pep 11 | LVD-PSEQ-A-Coronavirus Pep 11 | ENQKLIANTEVETPIRNEHYEECSCYQYIKANSKFIGITE |

### Description of Sequences Listed in Table 3

SEQ ID NO: 17. TB Pep 01- MTB 16.3HSP Conserved Region (CR).

SEQ ID NO: 18-23. PGN epitopes and MTB 16.3HSP (CR) with and without a T cell epitope. SEQ ID NO: 24 and 25. PGN and LTA peptides with a T cell epitope.

SEQ ID NO: 26. Coronavirus RNA polymerase and influenza matrix and neuraminidase (NA) peptides, with a T cell epitope.

SEQ ID NO: 27-28. Influenza peptides - 3 (Hemagglutinin, HA), 6 (HA), and 10 (NA).

SEQ ID NO: 29-32. Coronavirus peptides with and without a T cell epitope.

SEQ ID NO: 33-34. Influenza peptides - 52 and 53.

SEQ ID NO: 35. Influenza peptide 57.

SEQ ID NO: 36. Coronavirus spike protein epitope and influenza matrix and NA peptides with a T cell epitope.

### Example 5

### Composite Peptide Vaccines for Influenza and Other Viruses

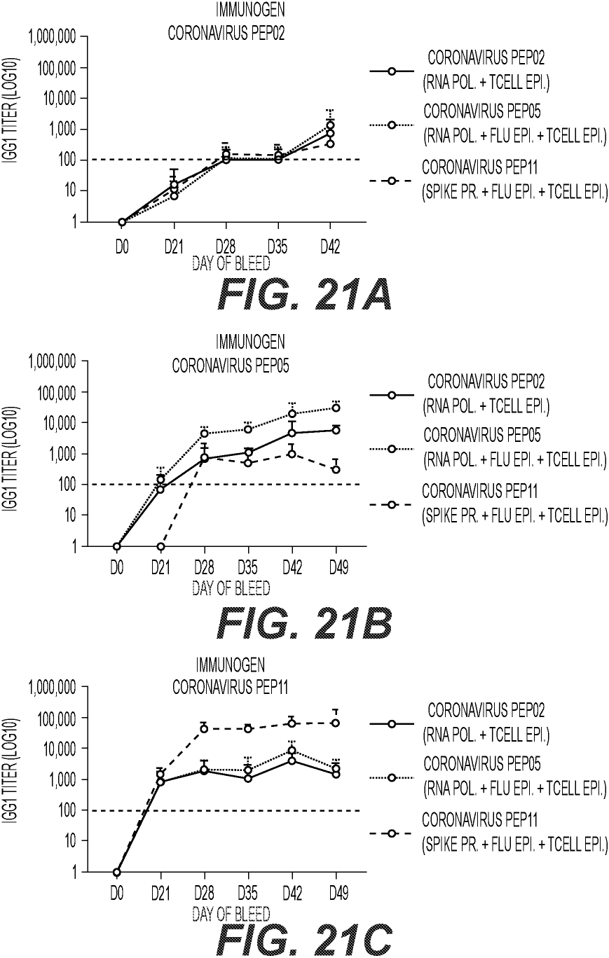

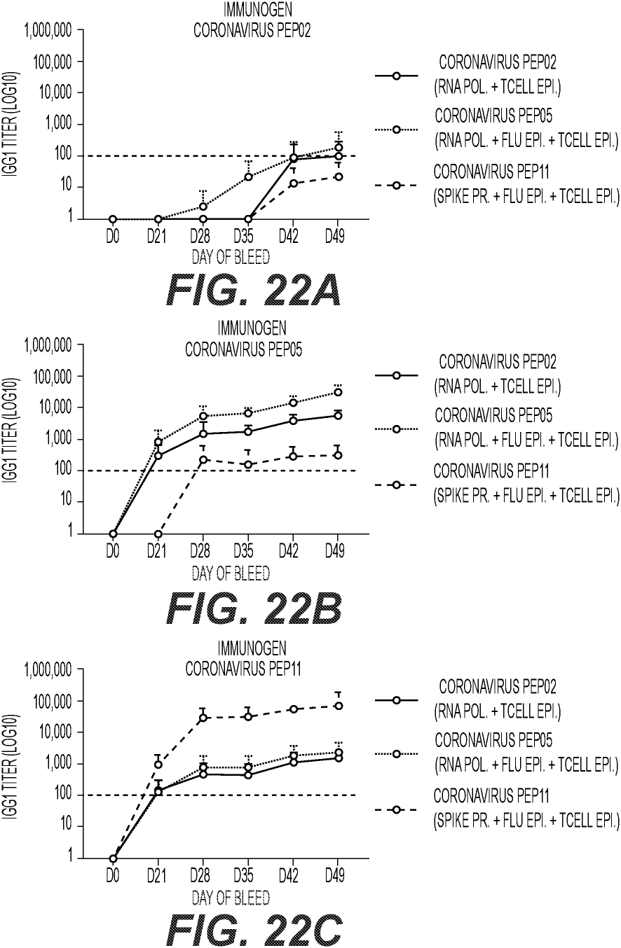

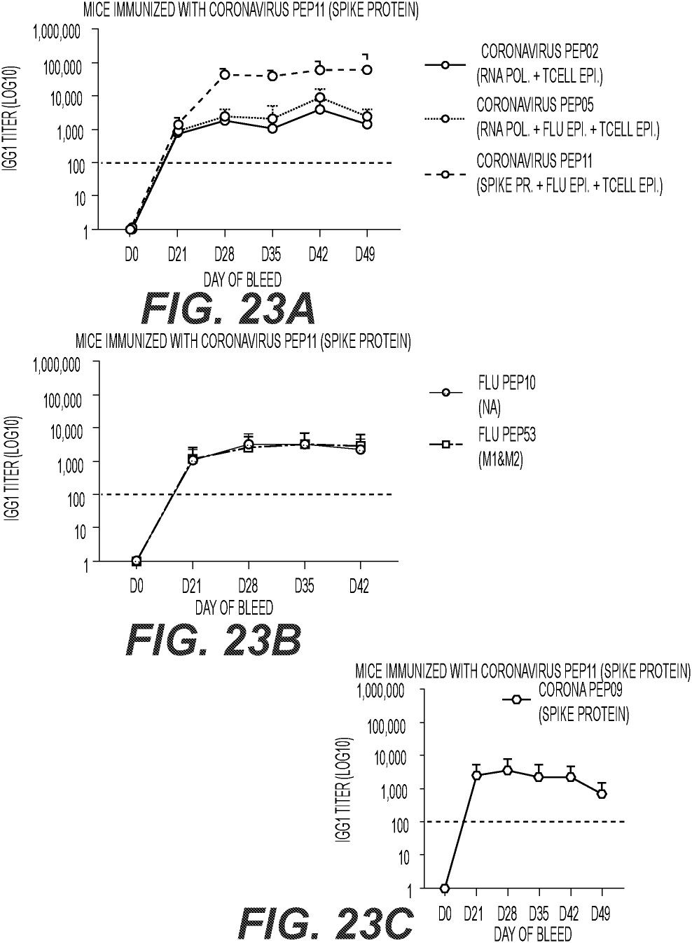

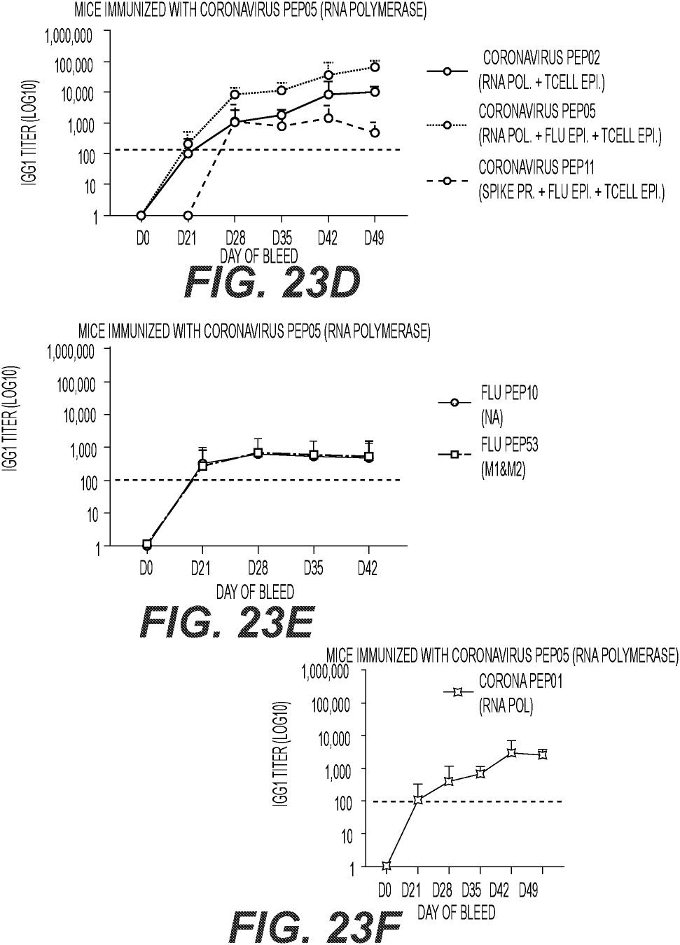

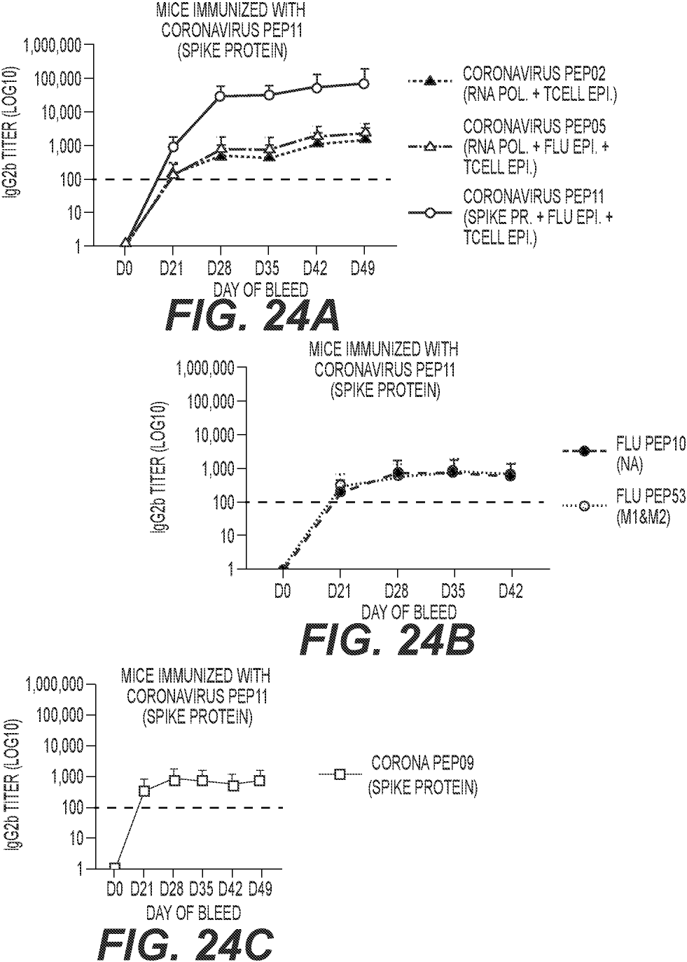

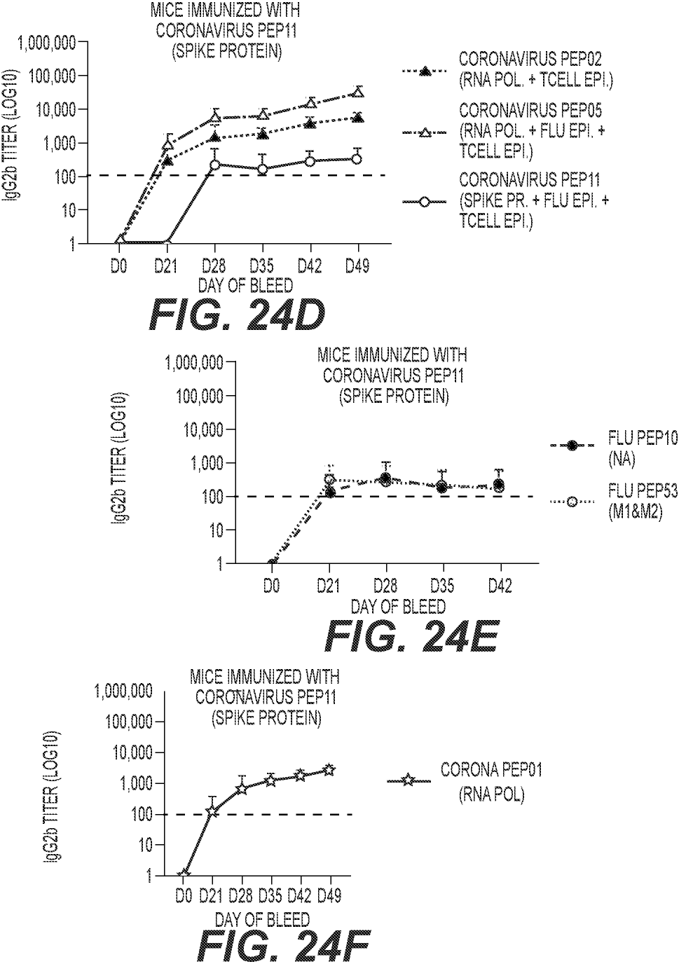

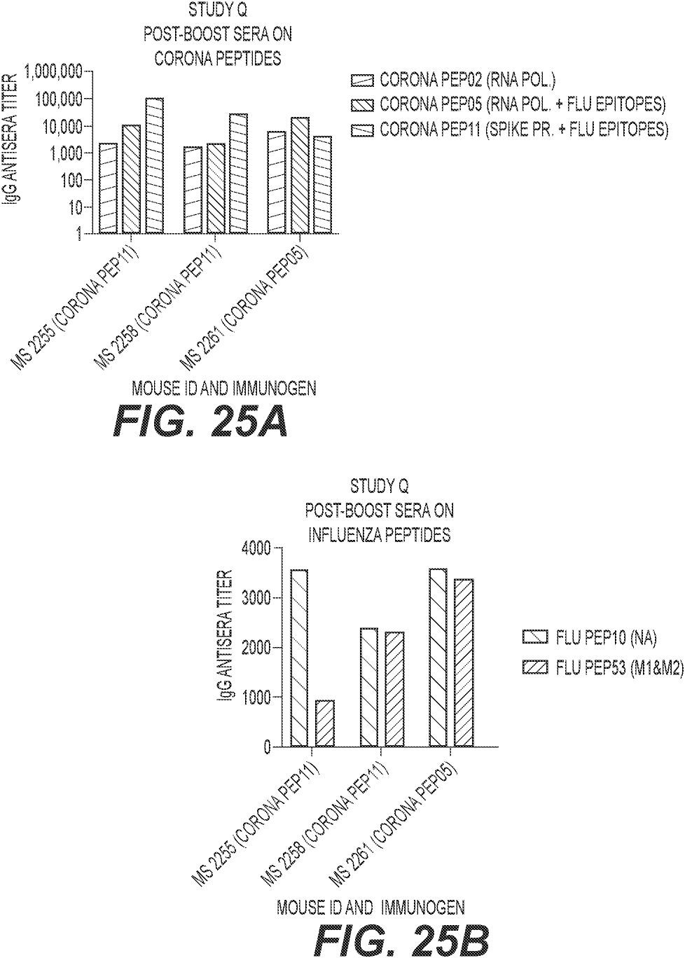

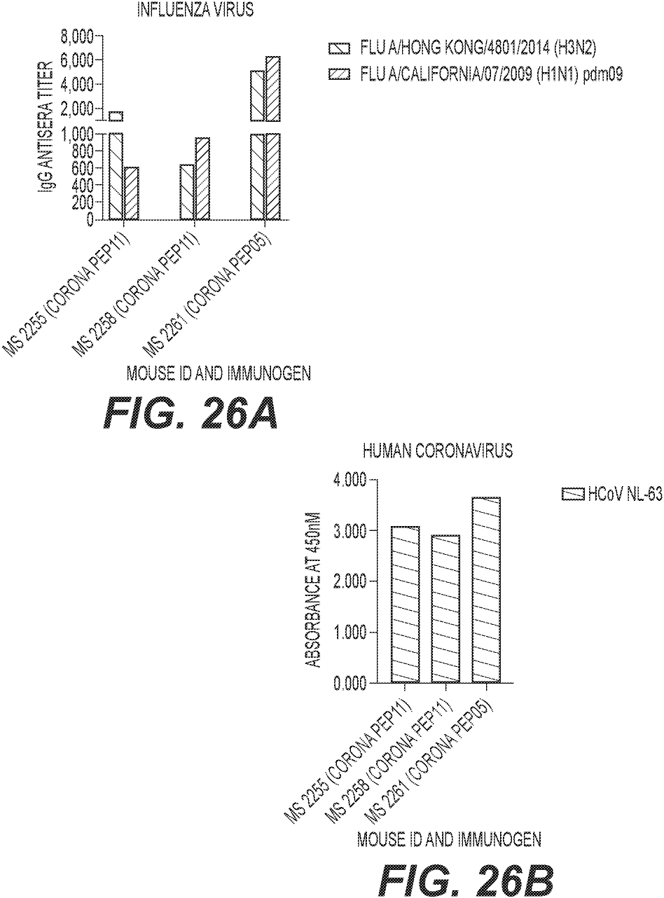

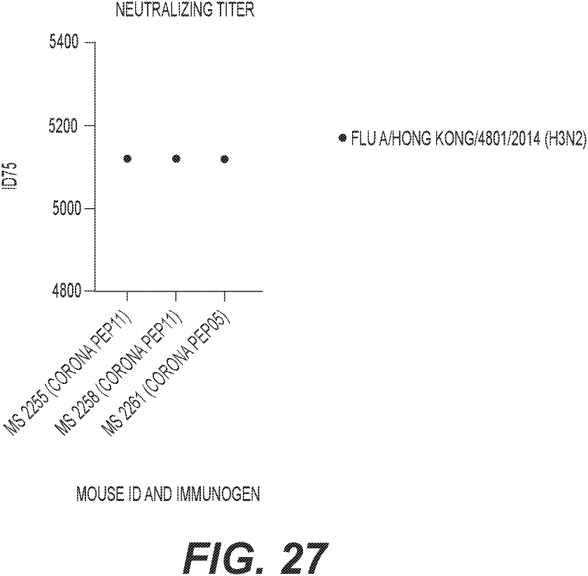

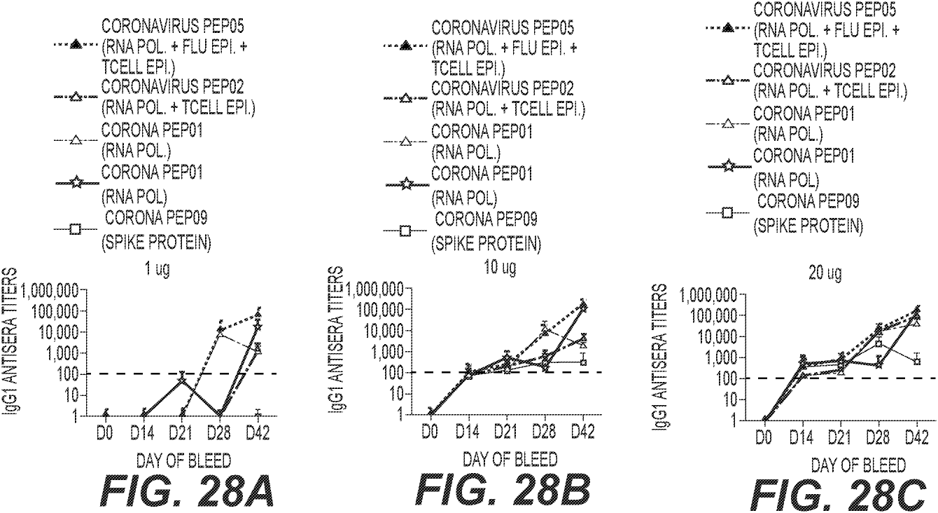

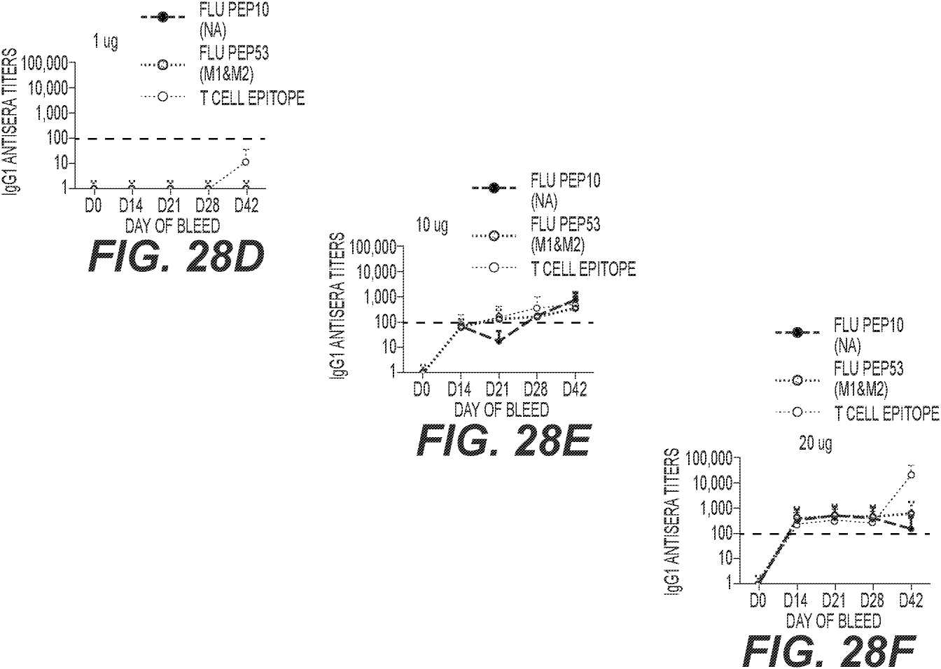

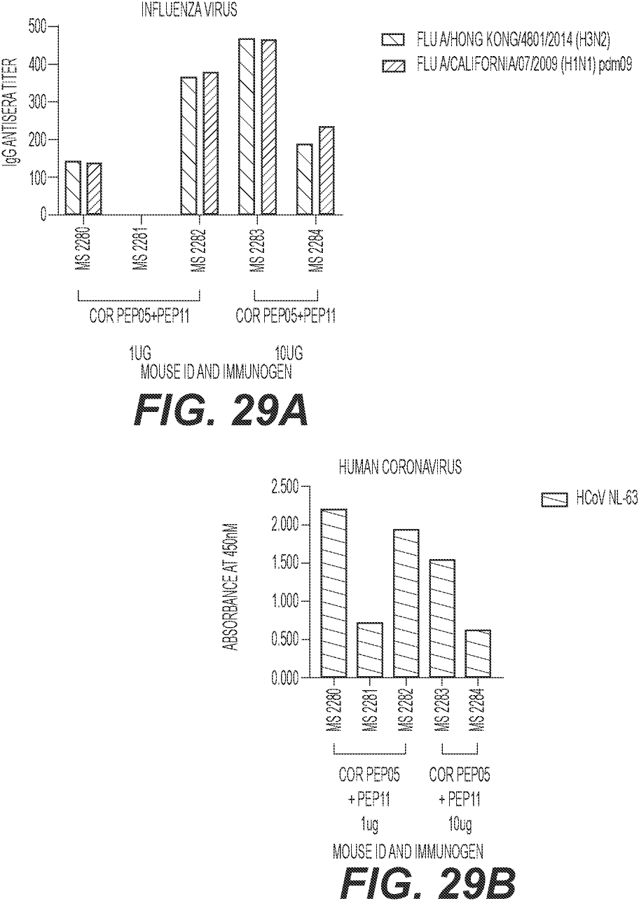

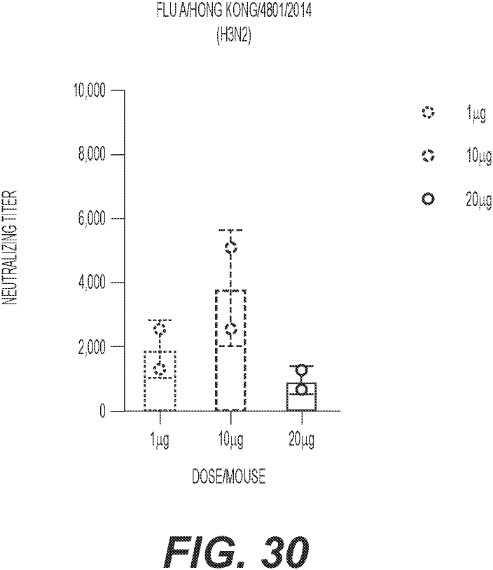

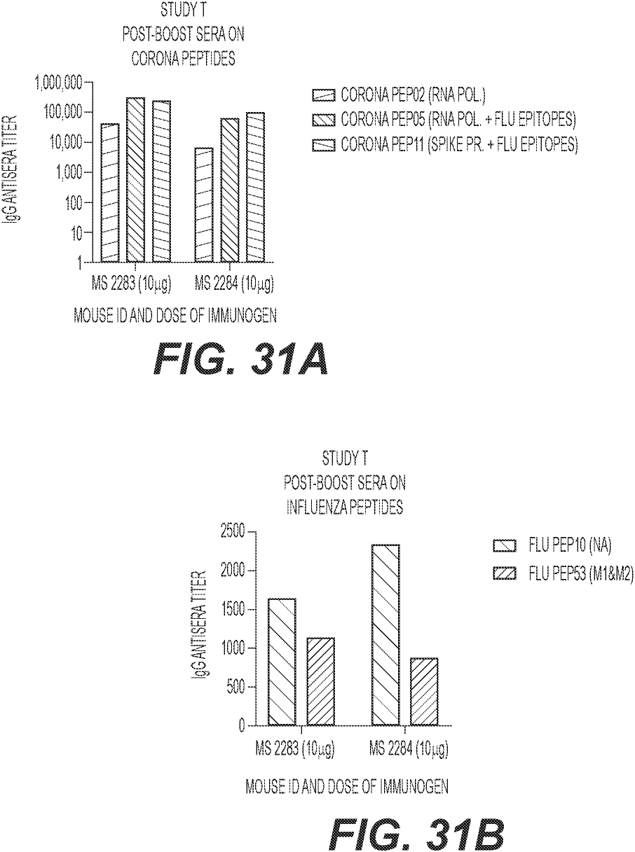

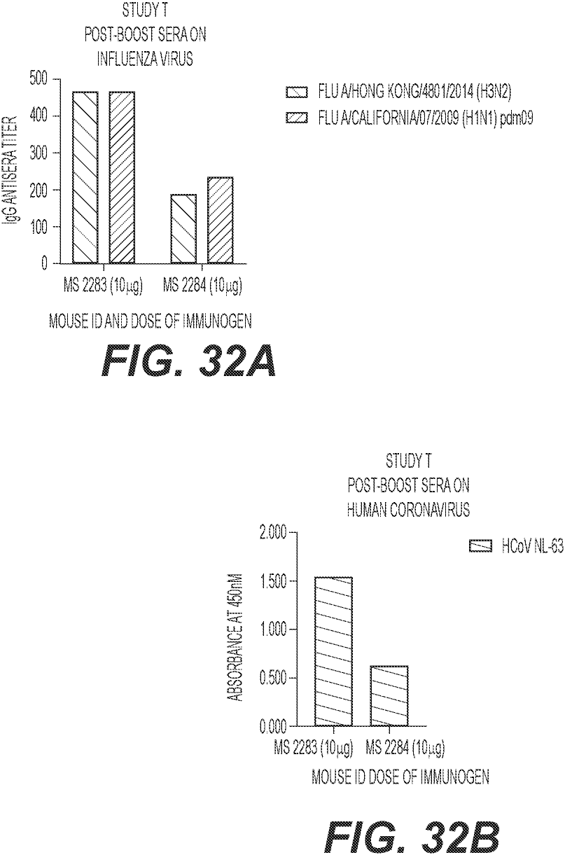

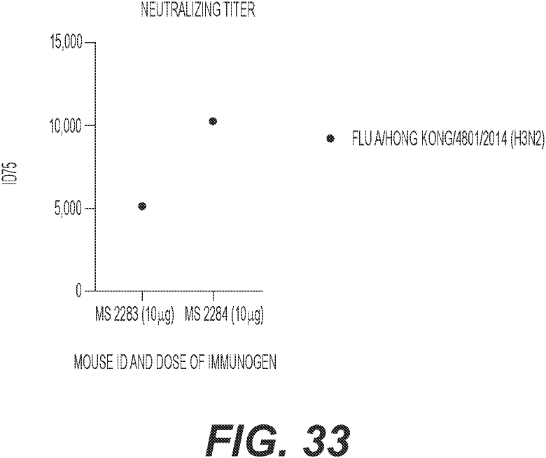

An influenza composite vaccine comprising small-conserved epitopes such as HA, NA, or matrix peptide sequences induce broadly neutralizing antibodies across Group 1 and 2 Influenza A viruses. Combining one or more of these peptides with one or more small-conserved peptide sequences from two or more viruses (such as influenza and coronavirus) provides a prototype composite virus peptide vaccine that broadens the vaccine’s prevention or treatment capabilities to include more than one virus (Table 4). Combined influenza and coronavirus composite peptide vaccine antigens were synthesized and included the conserved influenza matrix and NA peptides plus the conserved coronavirus polymerase peptide (Cor Pep 05), or spike protein conserved sequence (Cor Pep 11) and a T cell epitope sequence (Table 4). The polymerase conserved epitope was also sequenced alone with the T cell epitope (Cor Pep 02). Mice were immunized with one, or more of these peptides formulated with ADDAVAX™ adjuvant and given by either subcutaneous (SQ) injection at a dose of 20 µg,or Intradermal (ID) injection at 1 µg,10 µg,or 20 µgon days 0, 21 and 35. Robust serum IgG1 and IgG2b antibodies were induced to the conserved influenza and coronavirus epitopes. In addition, the serum antibodies induced by both SQ (Study Q: FIGS. 21-27) and ID immunization (Study T: FIGS. 28-33) bound to both live influenza and coronavirus and were strongly neutralizing (FIGS. 21-33).

TABLE 4

Microbial Peptides and Composite Peptide Antigens

| SEQ ID NO | Peptide number | Peptide ID | Peptide Sequence |
|---|---|---|---|
| 37 | CorPep01 | LVD-PSEQ-A-Cor Pep 01 | WDYPKCDRA |
| 38 | CorPep02 | LVD-PSEQ-A-Cor Pep 02 | WDYPKCDRAQYIKANSKFIGITE |
| 39 | CorPep05 | LVD-PSEQ-A-Cor Pep 05 | WDYPKCDRATEVETPIRNEHYEECSCYQYIKANSKFIGITE |
| 40 | CorPep09 | LVD-PSEQ-A-Cor Pep 09 | ENQKLIAN |
| 41 | CorPep11 | LVD-PSEQ-A-Cor Pep 11 | ENQKLIANTEVETPIRNEHYEECSCYQYIKANSKFIGITE |

### Description of Sequences Listed in Table 4

SEQ ID NO: 37. RNA polymerase region, non-spike.

SEQ ID NO: 38. Conserved regions from the RNA polymerase, Tetanus T-cell epitope.

SEQ ID NO: 39. Conserved regions from the RNA polymerase + Flu Pep53 (M2), Flu Pep10, Tetanus T-cell epitope.

SEQ ID NO: 40. Epitopes on spike protein.

SEQ ID NO: 41. Conserved SARS epitopes, Flu Pep53 (M2), Flu Pep10, Tetanus T-cell epitope.

Other embodiments and uses of the invention will be apparent to those skilled in the art from consideration of the specification and practice of the invention disclosed herein. All references cited herein, including all publications and U.S. and foreign patents and patent applications, are specifically and entirely incorporated by reference including U.S. Pat. No. 9,821,047 entitled “Enhancing Immunity to Tuberculosis,” which issued Nov. 21, 2017, U.S. Pat. No. 9,598.462 entitled “Composite Antigenic Sequences and Vaccines” which issued Mar. 21, 2017, U.S. Pat. No. 10,004,799 entitled “Composite Antigenic Sequences and Vaccines” which issued Jun. 26, 2018, U.S. Pat. No. 8,652,782 entitled “Compositions and Method for Detecting, Identifying and Quantitating Mycobacterial-Specific Nucleic Acid,” which issued Feb. 18, 2014, U.S. Pat. No. 9,481,912 entitled “Compositions and Method for Detecting, Identifying and Quantitating Mycobacterial-Specific Nucleic Acid,” which issued Nov. 1, 2016, U.S. Pat. No. 8,821,885 entitled “Immunogenic Compositions and Methods,” which issued Sep. 2, 2014, U.S. Application Publication No. 2021/0246174 entitled Immunogenic Compositions to Treat and Prevent Microbial Infections published Aug. 12, 2021, U.S. Application Publication No. 2022/0118079 entitled Immunogenic Antigens published Apr. 21, 2022, and U.S. Application Publication No. 2022/0280634 entitled Vaccines for the Treatment and Prevention of Zoonotic Infections published Sep. 8, 2022, and all corresponding U.S. Provisional and continuation applications relating to any of the foregoing patents. The term comprising, wherever used, is intended to include the terms consisting and consisting essentially of. Furthermore, the terms comprising, including, containing and the like are not intended to be limiting. It is intended that the specification and examples be considered exemplary only with the true scope and spirit of the invention indicated by the following claims.

1. An immunogenic peptide comprised of a contiguous sequence of any one of the sequences of SEQ ID NOs 1-4, 18-24, or a combination thereof.

2. The peptide of claim 1, wherein the contiguous sequence further includes one or more of the sequences selected from the group consisting of the sequences of SEQ ID NOs 5-17 and 25-41.

3. The peptide of claim 1, which contains a sequence of a viral antigen, a bacterial antigen, a parasitic antigen, a composite antigen, or a combination thereof.

4. The peptide of claim 3, wherein the bacterial antigen comprises an antigen of a gram-positive microorganism, a gram-negative microorganism, both gram-positive and gram-negative microorganisms, or an acid-fast microorganism.

5. The peptide of claim 1, which contains the sequence of a T-cell stimulating epitope.

6. The peptide of claim 1, which contains the sequence of a composite epitope.

7. The peptide of claim 1, wherein the composite epitope comprises a bacterial or viral epitope.

8. A nucleic acid that encodes the peptide of claim 1.

9. An immunogenic composition comprising the peptide of claim 1.

10. The immunogenic composition of claim 9, comprising one or more of a pharmaceutically acceptable carriers, a chemical agent, a diluent, an excipient, or an adjuvant.

11. The immunogenic composition of claim 10, wherein the pharmaceutically acceptable carrier, chemical agent, diluent, or excipient comprises water, fatty acids, lipids, polymers, carbohydrates, gelatin, solvents, saccharides, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, or a combination thereof.

12. The immunogenic composition of claim 10, wherein the adjuvant comprises alum, oil in water emulsion, amino acids, proteins, carbohydrates, Freund’s, a liposome, saponin, lipid A, squalene, liposomes adsorbed to aluminum hydroxide, liposomes containing QS21 saponin, liposomes containing QS21 saponin and adsorbed to aluminum hydroxide, liposomes containing saturated phospholipids, cholesterol, and/or monophosphoryl, ALFQ, ALFA, AS01, and/or modifications or derivatives thereof.

13. The immunogenic composition of claim 9, which is a vaccine.

14. An antibody that is reactive against the peptide of claim 1.

15. The antibody of claim 14, which comprises IgG, IgA, IgD, IgE, IgM or fragments or combinations thereof.

16. The antibody of claim 14, which is a polyclonal, a monoclonal, or a humanized antibody.

17. A hybridoma that expresses the monoclonal antibody of claim 16.

18. An antibody that is reactive against the peptide of claim 2.

19. The antibody of claim 18, which comprises IgG, IgA, IgD, IgE, IgM or fragments or combinations thereof.

20. The antibody of claim 18, which is a polyclonal, a monoclonal, or a humanized antibody.

21. A hybridoma that expresses the monoclonal antibody of claim 20.

22. An immunogenic peptide comprised of a contiguous sequence of any one of the sequences of SEQ ID NOs 25, 30, 32, 36, 38, 39, 41, or a combination thereof.

23. The peptide of claim 22, wherein the contiguous sequence further includes one or more of the sequences selected from the group consisting of the sequences of SEQ ID NOs 1-24, 26-29, 31, 33-35, 37, and 40.

24. The peptide of claim 22, which contains a sequence of a viral antigen, a bacterial antigen, a parasitic antigen, a composite antigen, or a combination thereof.

25. The peptide of claim 22, which contains the sequence of a T-cell stimulating epitope.

26. The peptide of claim 22, which contains the sequence of a composite epitope.

27. The peptide of claim 26, wherein the composite epitope comprises a bacterial or viral epitope.

28. A nucleic acid that encodes the peptide of claim 22.

29. An immunogenic composition comprising the peptide of claim 22.

30. The immunogenic composition of claim 29, comprising one or more of a pharmaceutically acceptable carriers, a chemical agent, a diluent, an excipient, or an adjuvant.

31. The immunogenic composition of claim 30, wherein the pharmaceutically acceptable carrier, chemical agent, diluent, or excipient comprises water, fatty acids, lipids, polymers, carbohydrates, gelatin, solvents, saccharides, buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, or a combination thereof.

32. The immunogenic composition of claim 30, wherein the adjuvant comprises alum, oil in water emulsion, amino acids, proteins, carbohydrates, Freund’s, a liposome, saponin, lipid A, squalene, liposomes adsorbed to aluminum hydroxide, liposomes containing QS21 saponin, liposomes containing QS21 saponin and adsorbed to aluminum hydroxide, liposomes containing saturated phospholipids, cholesterol, and/or monophosphoryl, ALFQ, ALFA, AS01, and/or modifications or derivatives thereof.

33. The immunogenic composition of claim 29, which is a vaccine.

34. An antibody that is reactive against the peptide of claim 22.

35. The antibody of claim 34, which comprises IgG, IgA, IgD, IgE, IgM or fragments or combinations thereof.

36. The antibody of claim 34, which is a polyclonal, a monoclonal, or a humanized antibody.

37. A hybridoma that expresses the monoclonal antibody of claim 36.

38. An antibody that is reactive against the peptide of claim 23.

39. The antibody of claim 38, which comprises IgG, IgA, IgD, IgE, IgM or fragments or combinations thereof.

40. The antibody of claim 38, which is a polyclonal, a monoclonal, or a humanized antibody.

41. A hybridoma that expresses the monoclonal antibody of claim 40.

42. A contiguous peptide sequence comprising an epitope of a bacterium and an epitope of a virus which includes one or more of the sequences selected from the group of sequences consisting of SEQ ID NOs. 1-41.

43. A contiguous peptide sequence comprising an epitope of a first bacterium and an epitope of a second bacterium, wherein the first bacterium and the second bacterium are of different serotypes, species or genera, which includes one or more of the sequences selected from the group of sequences consisting of SEQ ID NOs. 1-24.

44. A contiguous peptide sequence comprising an epitope of a first virus and an epitope of a second virus, wherein the first virus and the second virus are of different serotypes, species or genera, which includes one or more of the sequences selected from the group of sequences consisting of SEQ ID NOs. 25-41.


SEQUENCE LISTING (41 sequences)

SEQ ID NO: 1 - protein, 13 aa, synthetic construct
AEKAGGGGGAEKA

SEQ ID NO: 2 - protein, 19 aa, synthetic construct
AEKAEKAGGGGGAEKAEKA

SEQ ID NO: 3 - protein, 26 aa, synthetic construct
QYIKANSKFIGITEAEKAGGGGAEKA

SEQ ID NO: 4 - protein, 27 aa, synthetic construct
AEKAGGGGGAEKAQYIKANSKFIGITE

SEQ ID NO: 5 - protein, 4 aa, synthetic construct
AEKA

SEQ ID NO: 6 - protein, 9 aa, synthetic construct
AEKAGGGGG

SEQ ID NO: 7 - protein, 14 aa, Clostridium tetani
QYIKANSKFIGITE

SEQ ID NO: 8 - protein, 5 aa, synthetic construct
GGGGG

SEQ ID NO: 9 - protein, 20 aa, Mycobacterium tuberculosis
SEFAYGSFVRTVSLPVGADE

SEQ ID NO: 10 - protein, 42 aa, synthetic construct
SEFAYGSFVRTVSLPVGADEGNLFIAPWGVIHHPHYEECSCY

SEQ ID NO: 11 - protein, 12 aa, Mycobacterium tuberculosis
HSFKWLDSPRLR

SEQ ID NO: 12 - protein, 12 aa, Mycobacterium tuberculosis
ISLTEWSMWYRH

SEQ ID NO: 13 - protein, 15 aa, Staphylococcus sp.
WRMYFSHRHAHLRSP

SEQ ID NO: 14 - protein, 15 aa, Staphylococcus sp.
WHWRHRIPLQLAAGR

SEQ ID NO: 15 - protein, 22 aa, synthetic construct
GNLFIAPWGVIHHPHYEECSCY

SEQ ID NO: 16 - protein, 20 aa, Mycobacterium smegmatis
SEFAYGSFMRSVTLPPGADE

SEQ ID NO: 17 - protein, 20 aa, synthetic construct
SEFAYGSFVRTVSLPVGADE

SEQ ID NO: 18 - protein, 33 aa, synthetic construct
AEKAGGGGGAEKASEFAYGSFVRTVSLPVGADE

SEQ ID NO: 19 - protein, 47 aa, synthetic construct
AEKAGGGGGAEKASEFAYGSFVRTVSLPVGADEQYIKANSKFIGITE

SEQ ID NO: 20 - protein, 33 aa, synthetic construct
SEFAYGSFVRTVSLPVGADEAEKAGGGGGAEKA

SEQ ID NO: 21 - protein, 38 aa, synthetic construct
AEKAGGGGGSEFAYGSFVRTVSLPVGADEGGGGGAEKA

SEQ ID NO: 22 - protein, 52 aa, synthetic construct
AEKAGGGGGSEFAYGSFVRTVSLPVGADEGGGGGAEKAQYIKANSKFIGITE

SEQ ID NO: 23 - protein, 43 aa, synthetic construct
WRMYFSHRHAHLRSPGGGGGAEKAGGGGGQYIKANSKFIGITE

SEQ ID NO: 24 - protein, 52 aa, synthetic construct
WHWRHRIPLQLAGRAEKAGGGGGWRMYFSHRHAHLRSPQYIKANSKFIGITE

SEQ ID NO: 25 - protein, 41 aa, synthetic construct
WDYPKCDRATEVETPIRNEHYEECSCYQYIKANSKFIGITE

SEQ ID NO: 26 - protein, 7 aa, synthetic construct
GNLFIAP

SEQ ID NO: 27 - protein, 7 aa, synthetic construct
WGVIHHP

SEQ ID NO: 28 - protein, 8 aa, synthetic construct
HYEECSCY

SEQ ID NO: 29 - protein, 21 aa, synthetic construct
YFPLQSYGFQPTNGVGYQPYR

SEQ ID NO: 30 - protein, 35 aa, synthetic construct
YFPLQSYGFQPTNGVGYQPYRQYIKANSKFIGITE

SEQ ID NO: 31 - protein, 21 aa, synthetic construct
YQAGSTPCNGVEGFNCYFPLQ

SEQ ID NO: 32 - protein, 34 aa, synthetic construct
YQAGSTPCNGVEGFNCYFPLQYIKANSKFIGITE

SEQ ID NO: 33 - protein, 7 aa, synthetic construct
ETPIRNE

SEQ ID NO: 34 - protein, 10 aa, synthetic construct
TEVETPIRNE

SEQ ID NO: 35 - protein, 25 aa, synthetic construct
SLLTEVETPIRNEWGLLTEVETPIR

SEQ ID NO: 36 - protein, 40 aa, synthetic construct
ENQKLIANTEVETPIRNEHYEECSCYQYIKANSKFIGITE

SEQ ID NO: 37 - protein, 9 aa, synthetic construct
WDYPKCDRA

SEQ ID NO: 38 - protein, 23 aa, synthetic construct
WDYPKCDRAQYIKANSKFIGITE

SEQ ID NO: 39 - protein, 41 aa, synthetic construct; identical to SEQ ID NO: 25

SEQ ID NO: 40 - protein, 8 aa, synthetic construct
ENQKLIAN

SEQ ID NO: 41 - protein, 40 aa, synthetic construct; identical to SEQ ID NO: 36